(19) United States
(12) Patent Application Publication
(10) Pub. No.: US 2016/0302682 A1
(43) Pub. Date: Oct. 20, 2016

(54) NANOSCALE WIRE PROBES FOR THE BRAIN AND OTHER APPLICATIONS

(71) Applicant: PRESIDENT AND FELLOWS OF HARVARD COLLEGE, Cambridge, MA (US)

(72) Inventors: Charles M. Lieber, Lexington, MA (US); Chong Xie, Austin, TX (US); Jia Liu, Palo Alto, CA (US)

(73) Assignee: PRESIDENT AND FELLOWS OF HARVARD COLLEGE, Cambridge, MA (US)

(21) Appl. No.: 15/101,158

(22) PCT Filed: Dec. 2, 2014
(86) PCT No.: PCT/US14/68093

§ 371 Date: Jun. 2, 2016

Related U.S. Application Data
(60) Provisional application No. 61/911,294, filed on Dec. 3, 2013.

(51) Int. Cl. A61B 5/0478; A61B 5/042; G01N 27/414; A61B 5/00

(52) U.S. Cl. CPC A61B 5/0478; A61B 5/6868; A61B 5/042; G01N 27/4146; A61B 2562/0209; A61B 2562/0285; A61B 2562/125

(57) ABSTRACT

The present invention generally relates to nanoscale wires and, in particular, to probes comprising nanoscale wires for use in determining properties such as electrical and/or chemical properties, e.g., for insertion into biological tissue, such as the brain. The probe may be formed from relatively flexible materials such as polymers, and in some cases, the probes may comprises nanoscale wires or other electronic components. The probe may be cooled to a temperature that causes the probe to harden, e.g., to a temperature below a glass transition temperature, prior to insertion, to facilitate the insertion of the probe into the tissue.

### RELATED APPLICATIONS

This application claims the benefit of U.S. Provisional Patent Application Ser. No. 61/911,294, filed Dec. 3, 2013, entitled “Nanoscale Wire Probes for the Brain and other Applications,” by Lieber, et al., incorporated herein by reference.

### GOVERNMENT FUNDING

Research leading to various aspects of the present invention was sponsored, at least in part, by the National Institutes of Health, Grant No. 8DP1GM105379-05. The U.S. Government has certain rights in the invention.

### FIELD

The present invention generally relates to nanoscale wires and, in particular, to probes comprising nanoscale wires.

### BACKGROUND

Recent efforts in coupling electronics and tissues have focused on flexible, stretchable planar arrays that conform to tissue surfaces, or implantable microfabricated probes. These approaches have been limited in merging electronics with tissues while minimizing tissue disruption, because the support structures and electronic detectors are generally of a much larger scale than the extracellular matrix and the cells. Furthermore, planar arrays only probe the tissue near the device plane surface and cannot be used to study the internal 3-dimensional structure of the tissue. For example, probes using nanowire field-effect transistors have shown that electronic devices with nanoscopic features can be used to detect extra- and intracellular potentials from single cells, but are limited to only surface recording from 3-dimensional tissues and organs.

### SUMMARY

The present invention generally relates to nanoscale wires and, in particular, to probes comprising nanoscale wires for use in determining properties such as electrical and/or chemical properties. The subject matter of the present invention involves, in some cases, interrelated products, alternative solutions to a particular problem, and/or a plurality of different uses of one or more systems and/or articles.

In one aspect, the present invention is generally directed to a composition. In one set of embodiments the composition comprises an electrical network comprising nanoscale wires. In some cases, at least a portion of the electrical network is coated with a solid or a liquid having a melting point below 25° C. In certain embodiments, the electrical network is at a temperature of about −196° C. or less.

In another aspect, the present invention is generally directed to a method of inserting a probe, e.g., into a biological tissue, or other system. In one set of embodiments, the method includes acts of coating at least a portion of a probe with a liquid, the probe comprising a polymer and an electrical network comprising at least one nanoscale wire, exposing the probe to a temperature below the freezing point of the liquid and below the glass transition temperature of the polymer, whereby the liquid freezes, and optionally, inserting the probe into biological tissue.

The method, in another set of embodiments, includes acts of providing an electrically-sensing probe comprising a polymer, decreasing a cross-sectional area defined by the probe, relative to a direction of insertion, by at least about 25%, exposing the polymer to a temperature below the glass transition temperature of the polymer, and optionally, inserting the probe into a biological tissue.

In still another set of embodiments, the method includes acts of coating at least a portion of a probe with a biocompatible fluid, freezing at least a portion of the fluid coating on the probe, and optionally, inserting the probe into biological tissue.

In another aspect, the present invention encompasses methods of making one or more of the embodiments described herein, for example, a probe comprising one or more nanoscale wires. In still another aspect, the present invention encompasses methods of using one or more of the embodiments described herein, for example, a probe comprising one or more nanoscale wires.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1I illustrate certain probes in accordance with various embodiments of the invention;

FIGS. 2A-2I illustrate the preparation of probes in certain embodiments of the invention;

FIGS. 3A-3E illustrate the use of certain probes in a rat brain, in accordance with one embodiment of the invention;

FIG. 4 illustrates the design of a probe according to one embodiment of the invention;

FIG. 5 illustrates neurons around a probe, in another embodiment of the invention;

FIGS. 6A-6B illustrates extended bent portions on the probe, in certain embodiments of the invention; and

FIG. 7 schematically illustrates a cross-section of one embodiment of the invention.

### DETAILED DESCRIPTION

The present invention generally relates to nanoscale wires and, in particular, to probes comprising nanoscale wires for use in determining properties such as electrical and/or chemical properties, e.g., for insertion into biological tissue, such as the brain. The probe may be formed from relatively flexible materials such as polymers, and in some cases, the probes may comprises nanoscale wires or other electronic components. The probe may be cooled to a temperature that causes the probe to harden, e.g., to a temperature below a glass transition temperature, prior to insertion, to facilitate the insertion of the probe into the tissue. To increase the amount of time available for insertion and/or prevent rapid heating of the probe, the probe may also be coated with a liquid that is frozen on the probe. In addition, the liquid may, in some embodiments, be used to alter or collapse the probe prior to insertion. Other embodiments are generally directed to systems and methods of making, using, or promoting such probes, kits involving such probes, and the like.

One aspect of the present invention is generally directed to a probe for insertion into a tissue, or other material. The probe can be fully or partially inserted into the tissue or other material. The probe may be used to determine a property of the tissue or other material, and/or provide an electrical signal to the tissue, or other material. This may be achieved using one or more nanoscale wires on the probe. For example, in certain embodiments, a probe comprising nanoscale wires may be inserted into an electrically-active tissue, such as the heart or the brain, and the nanoscale wires may be used to determine electrical properties of the tissue, e.g., action potentials or other electrical activity. In some cases, the probe is relatively porous to allow cells, etc. to grow or migrate into the probe. This may be useful, for example, for long-term applications, for example, where the probe is to be inserted and used for days, weeks, months, or years within the tissue. For example, neurons or cardiac cells may be able to grow around and/or into the probe while it is inserted into the brain or the heart, e.g., over extended periods of time.

In some embodiments, a probe may be formed from one or more polymeric constructs and/or metal leads. In some cases, the probe is relatively small and may include components such as nanoscale wires. The probe may also be flexible and/or have a relatively open structure, e.g., an open porosity of at least about 30%, or other porosities as discussed herein. For instance, the probe may be formed from a plurality of nanoscale wires, connected by polymeric constructs and/or metal leads, forming a relatively large or open network, which can then be rolled to form a cylindrical or other 3-dimensional structure that is to be inserted into a subject. In some cases, the nanoscale wires may be distributed about the probe, e.g., in three dimensions, thereby allowing determining properties and/or stimulation of a tissue, etc. in three-dimensions. The probe can also be connected to an external electrical system, e.g., to facilitate use of the probe. Polymeric constructs, metal leads, nanoscale wires, the structure of the probe, and various properties of the probes are all discussed in additional detail below.

Due to the flexible nature of the probe, in some embodiments, a probe containing polymer constructs may be cooled to a temperature below the glass transition temperature of the polymer constructs. At such temperatures, the properties of the polymer constructs may change, e.g., such that the polymer enters a “glassy” state rather than a “rubbery” state. Accordingly, the probe may harden, which may be useful to facilitate insertion of the probe, i.e., without significant physical distortion that would occur if the probe were still flexible. However, upon warming of the probe to temperatures above the glass transition temperature (e.g., back to ambient temperatures (around 25° C.) or body temperatures (around 37° C.), the polymer constructs may enter the “rubbery” state and thereby become more flexible.

Any suitable technique may be used to cool the probe, or at least a portion of the probe, to temperatures below the glass transition temperature. For example, the probe may be exposed to cold air, a refrigerator (typically about 4° C.), a freezer (typically about −20° C.), a deep freezer (typically about −40° C. or about −80° C.), dry ice (−78.5° C.), or liquid nitrogen (−196° C.), etc. Other suitable cooling temperatures and techniques may also be used in some cases. Those of ordinary skill in the art will be able to readily ascertain the glass transition temperature of a polymer, and suitable techniques for reaching temperatures below this temperature.

In one aspect, the probe is first exposed to a liquid, which may be used to coat or surround at least a portion of the probe. The probe is then cooled, e.g., by exposure to liquid nitrogen or other cooling techniques such as is discussed herein, which may freeze at least some of the liquid coating the probe. Such a frozen coating of liquid on the probe may increase the amount of heat energy that would be needed to increase the temperature of the probe to a temperature above the glass transition temperature. Thus, the coating may increase the amount of time available for the probe to be inserted into a biological tissue before the probe becomes flexible. However, after warming up (e.g., to the temperature of the biological tissue), the probe may become more flexible. For example, a polymer may return to a “rubbery” state from a “glassy” state after insertion, such that the probe is only temporarily rigidified for the insertion process. In some cases, the probe may be repeatedly exposed to the liquid, e.g., to build up a series of coatings surround at least a portion of the probe.

In some cases, the liquid may be one that has a high relative heat capacity. Thus, more energy would be needed to heat or melt the frozen liquid on the probe. In addition, in certain embodiments, the liquid may be chosen to be relatively biocompatible or inert. Thus, upon melting (e.g., within the tissue), the liquid would not create any adverse reactions within the tissue. Non-limiting examples of suitable liquids include water, saline, water containing sodium chloride (for example, at physiological concentrations or osmolarities), cell media, or the like.

The liquid may be coated onto the probe using any suitable technique. Examples include immersion or insertion of the probe into the liquid (e.g., a pool of liquid), or the liquid may be sprayed or painted onto the probe, etc. After coating, the probe (with the coating) may be cooled as previously discussed, which may cause the liquid on the probe to enter a solid or frozen state.

In addition, in one set of embodiments, the probe may altered prior to exposure to the liquid and/or prior to cooling the probe. For example, the probe may be altered from a first configuration to a second configuration, e.g., by folding or other mechanical processes. The second configuration may be more strained than the first configuration, i.e., such that in the absence of external forces, the probe returns to the first configuration. However, in some cases, the probe is exposed to liquid and/or cooler temperatures, as discussed above, prior to the probe being able to return into the first configuration. In effect, the probe is temporarily “frozen” in the second configuration. After insertion of the probe into tissue, as discussed above, the probe may return to warmer temperatures, and the strain of the second configuration may thereby cause the probe to return (or at least partially return) to the first configuration. Thus, for example, a probe may be collapsed into a smaller configuration for insertion into a tissue and temporarily “frozen” in that configuration, where the probe subsequently returns (or at least partially) returns to its original configuration after being heated by the tissue.

For example, the probe may be exposed to a liquid having a relatively high surface tension, in some aspects of the invention, which may be used to decrease the cross-sectional area of the probe, relative to the direction of insertion into the tissue. For example, the probe may have the shape of a cylindrical roll, which may be pulled tighter upon insertion into a suitable liquid. Thus, for example, the liquid may cause the probe to adopt a smaller cross-sectional area, e.g., a decrease by at least about 5%, at least about 10%, at least about 15%, at least about 20%, at least about 25%, at least about 30%, at least about 40%, at least about 50%, at least about 60%, at least about 75%, etc., relative to the initial cross-sectional area. The liquid may be the same or different as the coating liquid. For example, in one set of embodiments, the liquid is saline.

In certain aspects, the probe itself may be defined by an electrical network comprising nanoscale wires and conductive pathways in electrical communication with the nanoscale wires. In some cases, at least some of the conductive pathways may also provide mechanical strength to the probe, and/or there may be polymeric or metal constructs that are used to provide mechanical strength to the probe. The probe may be planar or substantially define a plane, or the probe may be non-planar or curved (i.e., a surface that can be characterized as having a finite radius of curvature). The probe may also be flexible in some cases, e.g., the probe may be able to bend or flex. For example, a probe may be bent or distorted by a volumetric displacement of at least about 5%, about 10%, or about 20% (relative to the undisturbed volume), without causing cracks and/or breakage within the probe. For example, in some cases, the probe can be distorted such that about 5%, about 10%, or about 20% of the mass of the probe has been moved outside the original surface perimeter of the probe, without causing failure of the probe (e.g., by breaking or cracking of the probe, disconnection of portions of the electrical network, etc.). In some cases, the probe may be bent or flexed as described above by an ordinary human being without the use of tools, machines, mechanical device, excessive force, or the like. A flexible probe may be more biocompatible due to its flexibility, and the probe may be treated as previously discussed to facilitate its insertion into a tissue.

In addition, the probe may be non-planar in some cases, e.g., curved as previously discussed. For example, the probe may be substantially U-shaped or cylindrical, and/or have a shape and/or size that is similar to a hypodermic needle. In some embodiments, the probe may be generally cylindrical with a maximum outer diameter of no more than about 5 mm, no more than about 4 mm, no more than about 3 mm, no more than about 2 mm, no more than about 1 mm, no more than about 0.9 mm, no more than about 0.8 mm, no more than about 0.7 mm, no more than about 0.6 mm, no more than about 0.5 mm, no more than about 0.4 mm, no more than about 0.3 mm, or no more than about 0.2 mm.

In certain aspects, the probe may contain one or more polymeric constructs. The polymeric constructs typically comprise one or more polymers, e.g., photoresists, biocompatible polymers, biodegradable polymers, etc., and optionally may contain other materials, for example, metal leads or other conductive pathway materials. The polymeric constructs may be separately formed then assembled into the probe, and/or the polymeric constructs may be integrally formed as part of the probe, for example, by forming or manipulating (e.g. folding, rolling, etc.) the polymeric constructs into a 3-dimensional structure that defines the probe.

In one set of embodiments, some or all of the polymeric constructs have the form of fibers or ribbons. For example, the polymeric constructs may have one dimension that is substantially longer than the other dimensions of the polymeric construct. The fibers can in some cases be joined together to form a “network” or “mesh” of fibers that define the probe. For example, a probe may contain a plurality of fibers that are orthogonally arranged to form a regular network of polymeric constructs. However, the polymeric constructs need not be regularly arranged. The polymer constructs may have the form of fibers or other shapes. In general, any shape or dimension of polymeric construct may be used to form a probe.

In one set of embodiments, some or all of the polymeric constructs have a smallest dimension or a largest cross-sectional dimension of less than about 5 micrometers, less than about 4 micrometers, less than about 3 micrometers, less than about 2 micrometers, less than about 1 micrometer, less than about 700 nm, less than about 600 nm, less than about 500 nm, less than about 300 nm, less than about 200 nm, less than about 100 nm, less than about 80 nm, less than about 50 nm, less than about 30 nm, less than about 10 nm, less than about 5 nm, less than about 2 nm, etc. A polymeric construct may also have any suitable cross-sectional shape, e.g., circular, square, rectangular, polygonal, elliptical, regular, irregular, etc. Examples of methods of forming polymeric constructs, e.g., by lithographic or other techniques, are discussed below.

In one set of embodiment, the polymeric constructs can be arranged such that the probe is relatively porous, e.g., such that cells can penetrate into the probe after insertion of the probe. For example, in some cases, the polymeric constructs may be constructed and arranged within the probe such that the probe has an open porosity of at least about 30%, at least about 40%, at least about 50%, at least about 60%, at least about 70%, at least about 75%, at least about 80%, at least about 85%, at least about 90%, at least about 95%, at least about 97, at least about 99%, at least about 99.5%, or at least about 99.8%. The “open porosity” is generally described as the volume of empty space within the probe divided by the overall volume defined by the probe, and can be thought of as being equivalent to void volume. Typically, the open porosity includes the volume within the probe to which cells can access. In some cases, the probe does not contain significant amounts of internal volume to which the cells are incapable of addressing, e.g., due to lack of access and/or pore access being too small.

In some cases, a “two-dimensional open porosity” may also be defined, e.g., of a probe that is subsequently formed or manipulated into a 3-dimensional structure. The two-dimensional open porosities of a probe can be defined as the void area within the two-dimensional configuration of the probe (e.g., where no material is present) divided by the overall area of probe, and can be determined before or after the probe has been formed into a 3-dimensional structure. Depending on the application, a probe may have a two-dimensional open porosity of at least about 30%, at least about 40%, at least about 50%, at least about 60%, at least about 70%, at least about 75%, at least about 80%, at least about 85%, at least about 90%, at least about 95%, at least about 97, at least about 99%, at least about 99.5%, or at least about 99.8%, etc.

Another method of generally determining the two-dimensional porosity of the probe is by determining the areal mass density, i.e., the mass of the probe divided by the area of one face of the probe (including holes or voids present therein). Thus, for example, in another set of embodiments, the probe may have an areal mass density of less than about 100 micrograms/cm², less than about 80 micrograms/cm², less than about 60 micrograms/cm², less than about 50 micrograms/cm², less than about 40 micrograms/cm², less than about 30 micrograms/cm², or less than about 20 micrograms/cm².

The porosity of a probe can be defined by one or more pores. Pores that are too small can hinder or restrict cell access. Thus, in one set of embodiments, the probe may have an average pore size of at least about 100 micrometers, at least about 200 micrometers, at least about 300 micrometers, at least about 400 micrometers, at least about 500 micrometers, at least about 600 micrometers, at least about 700 micrometers, at least about 800 micrometers, at least about 900 micrometers, or at least about 1 mm. However, in other embodiments, pores that are too big may prevent cells from being able to satisfactorily use or even access the pore volume. Thus, in some cases, the probe may have an average pore size of no more than about 1.5 mm, no more than about 1.4 mm, no more than about 1.3 mm, no more than about 1.2 mm, no more than about 1.1 mm, no more than about 1 mm, no more than about 900 micrometers, no more than about 800 micrometers, no more than about 700 micrometers, no more than about 600 micrometers, or no more than about 500 micrometers. Combinations of these are also possible, e.g., in one embodiment, the average pore size is at least about 100 micrometers and no more than about 1.5 mm. In addition, larger or smaller pores than these can also be used in a probe in certain cases. Pore sizes may be determined using any suitable technique, e.g., through visual inspection (e.g., of microscope images), BET measurements, or the like.

In various embodiments, one or more of the polymers forming a polymeric construct may be a photoresist. While not commonly used in biological probes, photoresists are typically used in lithographic techniques, which can be used as discussed herein to form the polymeric construct. For example, the photoresist may be chosen for its ability to react to light to become substantially insoluble (or substantially soluble, in some cases) to a photoresist developer. For instance, photoresists that can be used within a polymeric construct include, but are not limited to, SU-8, S1805, LOR 3A, poly(methyl methacrylate), poly(methyl glutarimide), phenol formaldehyde resin (diazonaphthoquinone/novolac), diazonaphthoquinone (DNQ), Hoechst AZ 4620, Hoechst AZ 4562, Shipley 1400-17, Shipley 1400-27, Shipley 1400-37, or the like. These and many other photoresists are available commercially.

A polymeric construct may also contain one or more polymers that are biocompatible and/or biodegradable, in certain embodiments. A polymer can be biocompatible, biodegradable, or both biocompatible and biodegradable, and in some cases, the degree of biodegradation or biocompatibility depends on the physiological environment to which the polymer is exposed to.

Typically, a biocompatible material is one that does not illicit an immune response, or elicits a relatively low immune response, e.g., one that does not impair the probe or the cells therein from continuing to function for its intended use. In some embodiments, the biocompatible material is able to perform its desired function without eliciting any undesirable local or systemic effects in the subject. In some cases, the material can be incorporated into tissues within the subject, e.g., without eliciting any undesirable local or systemic effects, or such that any biological response by the subject does not substantially affect the ability of the material from continuing to function for its intended use. For example, in a probe, the probe may be able to determine cellular or tissue activity after insertion, e.g., without substantially eliciting undesirable effects in those cells, or undesirable local or systemic responses, or without eliciting a response that causes the probe to cease functioning for its intended use. Examples of techniques for determining biocompatibility include, but are not limited to, the ISO 10993 series of for evaluating the biocompatibility of medical devices. As another example, a biocompatible material may be implanted in a subject for an extended period of time, e.g., at least about a month, at least about 6 months, or at least about a year, and the integrity of the material, or the immune response to the material, may be determined. For example, a suitably biocompatible material may be one in which the immune response is minimal, e.g., one that does not substantially harm the health of the subject. One example of a biocompatible material is poly(methyl methacrylate). In some embodiments, a biocompatible material may be used to cover or shield a non-biocompatible material (or a poorly biocompatible material) from the cells or tissue, for example, by covering the material.

A biodegradable material typically degrades over time when exposed to a biological system, e.g., through oxidation, hydrolysis, enzymatic attack, phagocytosis, or the like. For example, a biodegradable material can degrade over time when exposed to water (e.g., hydrolysis) or enzymes. In some cases, a biodegradable material is one that exhibits degradation (e.g., loss of mass and/or structure) when exposed to physiological conditions for at least about a month, at least about 6 months, or at least about a year. For example, the biodegradable material may exhibit a loss of mass of at least about 10%, at least about 20%, at least about 30%, at least about 40%, at least about 50%, at least about 60%, at least about 70%, at least about 80%, or at least about 90%. In certain cases, some or all of the degradation products may be resorbed or metabolized, e.g., into cells or tissues. For example, certain biodegradable materials, during degradation, release substances that can be metabolized by cells or tissues. For instance, polylactic acid releases water and lactic acid during degradation.

Examples of such biocompatible and/or biodegradable polymers include, but are not limited to, poly(lactic-co-glycolic acid), polylactic acid, polyglycolic acid, poly(methyl methacrylate), poly(trimethylene carbonate), collagen, fibrin, polysaccharidic materials such as chitosan or glycosaminoglycans, hyaluronic acid, polycaprolactone, and the like.

The polymers and other components forming the probe can also be used in some embodiments to provide a certain degree of flexibility to the probe, which can be quantified as a bending stiffness per unit width of polymer construct. In various embodiments, the probe may have a bending stiffness of less than about 5 nN m, less than about 4.5 nN m, less than about 4 nN m, less than about 3.5 nN m, less than about 3 nN m, less than about 2.5 nN m, less than about 2 nN m, less than about 1.5 nN m, or less than about 1 nN m.

In some embodiments of the invention, the probe may also contain other materials in addition to the photoresists or biocompatible and/or biodegradable polymers described above. Non-limiting examples include other polymers, growth hormones, extracellular matrix protein, specific metabolites or nutrients, or the like. For example, in one of embodiments, one or more agents able to promote cell growth can be added to the probe, e.g., hormones such as growth hormones, extracellular matrix protein, pharmaceutical agents, vitamins, or the like. Many such growth hormones are commercially available, and may be readily selected by those of ordinary skill in the art based on the specific type of cell or tissue used or desired. Similarly, non-limiting examples of extracellular matrix proteins include gelatin, laminin, fibronectin, heparan sulfate, proteoglycans, entactin, hyaluronic acid, collagen, elastin, chondroitin sulfate, keratan sulfate, Matrigel™, or the like. Many such extracellular matrix proteins are available commercially, and also can be readily identified by those of ordinary skill in the art based on the specific type of cell or tissue used or desired.

As another example, in one set of embodiments, additional materials can be added to the probe, e.g., to control the size of pores within the probe, to promote cell adhesion or cell growth within the probe, to increase the structural stability of the probe, to control the flexibility of the probe, etc. For instance, in one set of embodiments, additional fibers or other suitable polymers may be added to the probe, e.g., electrospun fibers can be used as a secondary scaffold. The additional materials can be formed from any of the materials described herein, e.g., photoresists or biocompatible and/or biodegradable polymers, or other polymers described herein. As another non-limiting example, a glue such as a silicone elastomer glue can be used to control the shape of the probe.

In some cases, the probe can include a 2-dimensional structure that is formed into a final 3-dimensional structure, e.g., by folding or rolling the structure. It should be understood that although the 2-dimensional structure can be described as having an overall length, width, and height, the overall length and width of the structure may each be substantially greater than the overall height of the structure. The 2-dimensional structure may also be manipulated to have a different shape that is 3-dimensional, e.g., having an overall length, width, and height where the overall length and width of the structure are not each substantially greater than the overall height of the structure. For instance, the structure may be manipulated to increase the overall height of the material, relative to its overall length and/or width, for example, by folding or rolling the structure. Thus, for example, a relatively planar sheet of material (having a length and width much greater than its thickness) may be rolled up into a “tube,” such that the tube has an overall length, width, and height of relatively comparable dimensions).

Thus, for example, the 2-dimensional structure may comprise one or more nanoscale wires and one or more polymeric constructs formed into a 2-dimensional structure or network that is subsequently formed into a 3-dimensional structure. In some embodiments, the 2-dimesional structure may be rolled or curled up to form the 3-dimesional structure, or the 2-dimensional structure may be folded or creased one or more times to form the 3-dimesional structure. Such manipulations can be regular or irregular. In certain embodiments, as discussed herein, the manipulations are caused by pre-stressing the 2-dimensional structure such that it spontaneously forms the 3-dimensional structure, although in other embodiments, such manipulations can be performed separately, e.g., after formation of the 2-dimensional structure.

In some aspects, the probe may include one or more metal leads. The metal leads may provide mechanical support, and/or one or more metal leads can be used within a conductive pathway to a nanoscale wire. The metal lead may directly physically contact the nanoscale wire and/or there may be other materials between the metal lead and the nanoscale wire that allow electrical communication to occur. Metal leads are useful due to their high conductance, e.g., such that changes within electrical properties obtained from the conductive pathway can be related to changes in properties of the nanoscale wire, rather than changes in properties of the conductive pathway. However, it is not a requirement that only metal leads be used, and in other embodiments, other types of conductive pathways may also be used, in addition or instead of metal leads.

A wide variety of metal leads can be used, in various embodiments of the invention. As non-limiting examples, the metals used within a metal lead may include aluminum, gold, silver, copper, molybdenum, tantalum, titanium, nickel, tungsten, chromium, palladium, as well as any combinations of these and/or other metals. In some cases, the metal can be chosen to be one that is readily introduced into the probe, e.g., using techniques compatible with lithographic techniques. For example, in one set of embodiments, lithographic techniques such as e-beam lithography, photolithography, X-ray lithography, extreme ultraviolet lithography, ion projection lithography, etc. may be used to layer or deposit one or more metals on a substrate. Additional processing steps can also be used to define or register the metal leads in some cases. Thus, for example, the thickness of a metal layer may be less than about 5 micrometers, less than about 4 micrometers, less than about 3 micrometers, less than about 2 micrometers, less than about 1 micrometer, less than about 700 nm, less than about 600 nm, less than about 500 nm, less than about 300 nm, less than about 200 nm, less than about 100 nm, less than about 80 nm, less than about 50 nm, less than about 30 nm, less than about 10 nm, less than about 5 nm, less than about 2 nm, etc. The thickness of the layer may also be at least about 10 nm, at least about 20 nm, at least about 40 nm, at least about 60 nm, at least about 80 nm, or at least about 100 nm. For example, the thickness of a layer may be between about 40 nm and about 100 nm, between about 50 nm and about 80 nm.

In some embodiments, more than one metal can be used within a metal lead. For example, two, three, or more metals may be used within a metal lead. The metals may be deposited in different regions or alloyed together, or in some cases, the metals may be layered on top of each other, e.g., layered on top of each other using various lithographic techniques. For example, a second metal may be deposited on a first metal, and in some cases, a third metal may be deposited on the second metal, etc. Additional layers of metal (e.g., fourth, fifth, sixth, etc.) may also be used in some embodiments. The metals can all be different, or in some cases, some of the metals (e.g., the first and third metals) may be the same. Each layer may independently be of any suitable thickness or dimension, e.g., of the dimensions described above, and the thicknesses of the various layers can independently be the same or different.

If dissimilar metals are layered on top of each other, they may be layered in some embodiments in a “stressed” configuration (although in other embodiments they may not necessarily be stressed). As a specific non-limiting example, chromium and palladium can be layered together to cause stresses in the metal leads to occur, thereby causing warping or bending of the metal leads. The amount and type of stress may also be controlled, e.g., by controlling the thicknesses of the layers. For example, relatively thinner layers can be used to increase the amount of warping that occurs.

Without wishing to be bound by any theory, it is believed that layering metals having a difference in stress (e.g., film stress) with respect to each other may, in some cases, cause stresses within the metal, which can cause bending or warping as the metals seek to relieve the stresses. In some embodiments, such mismatches are undesirable because they could cause warping of the metal leads and thus, the probe. However, in other embodiments, such mismatches may be desired, e.g., so that the probe can be intentionally deformed to form a 3-dimensional structure, as discussed below. In addition, in certain embodiments, the deposition of mismatched metals within a lead may occur at specific locations within the probe, e.g., to cause specific warpings to occur, which can be used to cause the probe to be deformed into a particular shape or configuration. For example, a “line” of such mismatches can be used to cause an intentional bending or folding along the line of the probe.

In addition, in one aspect, one or more nanoscale wires (e.g., a nanotube or a nanowire) may be held at an angle away from the surface of the probe, for example, by a suitable holding member. See, e.g., FIG. 6A, illustrating various bent portions on the probe, which may contain one or more nanoscale wires. Any method may be used to produce such bent portions, including those discussed in International Patent Application No. PCT/US2013/039228, filed May 2, 2013, entitled “Nanoscale Sensors for Intracellular and Other Applications,” by Lieber, et al., incorporated herein by reference in its entirety.

In some embodiments, the holding member comprises a polymer, such as a photoresist. For example, the photoresist can be chosen for its ability to react to light to become substantially insoluble (or substantially soluble, in some cases) to a photoresist developer. For instance, photoresists that may be used within a polymeric construct include, but are not limited to, SU-8, S1805, LOR 3A, poly(methyl methacrylate), poly(methyl glutarimide), phenol formaldehyde resin (diazonaphthoquinone/novolac), diazonaphthoquinone (DNQ), Hoechst AZ 4620, Hoechst AZ 4562, Shipley 1400-17, Shipley 1400-27, Shipley 1400-37, or the like. These and many other photoresists are available commercially. In some embodiments, one or more portions of the photoresist can be exposed to light (visible, UV, etc.), electrons, ions, X-rays, etc. (e.g., projected onto the photoresist), and the exposed portions may be etched away (e.g., using suitable etchants, plasma, etc.) to produce the pattern.

In some embodiments, only a portion of the nanoscale wire is held by the holding member, e.g., such that only one end of the nanoscale wire is supported by the holding member. For example, the nanoscale wire may comprise a free portion (not in physical contact with the holding member) and a held portion (in physical contact with the holding member), such that the free portion is at least about 10%, at least about 20%, at least about 30%, at least about 40%, at least about 50%, at least about 60%, at least about 70%, at least about 80%, or at least about 90% of the nanoscale wire. In addition, more than one free portion and/or held portion may be present in some embodiments.

The holding member may hold the nanoscale wire at any suitable angle away from the probe. For example, the angle can be about 10°, about 20°, about 30°, about 40°, about 50°, about 60°, about 70°, about 80°, or about 90° (i.e., vertically positioned relative to the probe). If more than one nanoscale wire is held by the holding member, the nanoscale wires can be held at the same or different angles.

The holding member may be angled away from the probe, in one set of embodiments, by depositing two or more dissimilar metals on the holding member that may warp or bend, thereby causing the holding member to warp away from the remainder of the probe. In some (but not all) embodiments, the metals can also be used for one or more electrodes, e.g., as discussed herein. As a specific non-limiting example, chromium and palladium may be layered or deposited on each other in such a way that stresses occur between the metals, thereby causing warping or bending. As another non-limiting example, copper and chromium may be layered or deposited on each other to cause warping or bending. The amount and type of stress can also be controlled, e.g., by controlling the thicknesses of the layers. For example, relatively thinner layers may be used to increase the amount of warping that occurs.

As specific examples, the thickness of a metal can be less than about 5 micrometers, less than about 4 micrometers, less than about 3 micrometers, less than about 2 micrometers, less than about 1 micrometer, less than about 700 nm, less than about 600 nm, less than about 500 nm, less than about 300 nm, less than about 200 nm, less than about 100 nm, less than about 80 nm, less than about 50 nm, less than about 30 nm, less than about 10 nm, less than about 5 nm, less than about 2 nm, etc. The thickness of the layer may also be at least about 10 nm, at least about 20 nm, at least about 40 nm, at least about 60 nm, at least about 80 nm, or at least about 100 nm. For example, the thickness of a metal can be between about 40 nm and about 100 nm, between about 50 nm and about 80 nm. In addition, the thicknesses of the two or more metals may independently be the same or different.

Furthermore, in some cases, longer lengths of metals may also be used to control the amount of bending or warping, in addition to and/or instead of controlling the thicknesses of the metals. For example, by using longer lengths in the holding member, larger angles and/or heights of the nanoscale wire, relative to the probe, may be achieved. For example, the effect of a relatively small deflection in two dissimilar metals may be geometrically increased due to longer lengths of metals that are bent or warped, even if at any one location, the amount of deflection or stress is relatively small.

In some embodiments, more than one conductive pathway may be used within the probe. For example, multiple conductive pathways can be used such that some or all of the nanoscale wires may be individually electronically addressable within the probe. However, in other embodiments, more than one nanoscale wire may be addressable by a particular conductive pathway. In addition, in some cases, other electronic components may also be present within the probe, e.g., as part of a conductive pathway or otherwise forming part of an electrical circuit. Examples include, but are not limited to, transistors such as field effect transistors, resistors, capacitors, inductors, diodes, integrated circuits, etc. In some cases, some of these may also comprise nanoscale wires.

In addition, in some cases, the conductive pathway and/or electronic components can be at least partially surrounded by or contained within one or more polymeric constructs used to form the probe. For example, a conductive pathway, such as a metal lead, may be “sandwiched” between two polymers (which can be the same or different from each other) that form a polymeric construct of the probe. Accordingly, in some embodiments, the conductive pathway may be relatively narrow. For example, the conductive pathway may have a smallest dimension or a largest cross-sectional dimension of less than about 5 micrometers, less than about 4 micrometers, less than about 3 micrometers, less than about 2 micrometers, less than about 1 micrometer, less than about 700 nm, less than about 600 nm, less than about 500 nm, less than about 300 nm, less than about 200 nm, less than about 100 nm, less than about 80 nm, less than about 50 nm, less than about 30 nm, less than about 10 nm, less than about 5 nm, less than about 2 nm, etc. The conductive pathway may have any suitable cross-sectional shape, e.g., circular, square, rectangular, polygonal, elliptical, regular, irregular, etc. As is discussed in detail below, such conductive pathways may be achieved using lithographic or other techniques.

A given conductive pathway within a probe may be in electrical communication with any number of nanoscale wires within a probe, depending on the embodiment. For example, a conductive pathway can be in electrical communication with one, two, three, or more nanoscale wires, and if more than one nanoscale wire is used within a given conductive pathway, the nanoscale wires may each independently be the same or different. Thus, for example, an electrical property of the nanoscale wire may be determined via the conductive pathway, and/or a signal can be propagated via the conductive pathway to the nanoscale wire. In addition, as previously discussed, some or all of the nanoscale wires may be in electrical communication with a surface of the probe via one or more conductive pathways. For example, in some cases, at least about 10%, at least about 20%, at least about 30%, at least about 40%, at least about 50%, at least about 60%, at least about 70%, at least about 80%, or at least about 90% of the nanoscale wires within the probe may be in electrical communication with one or more conductive pathways, or otherwise form portions of one or more electrical circuits extending externally of the probe. In some cases, however, not all of the nanoscale wires within a probe may be in electrical communication with one or more conductive pathways, e.g., by design, or because of inefficiencies within the fabrication process, etc.

The probe may include one or more nanoscale wires, which may be the same or different from each other, in accordance with various aspects of the invention. Non-limiting examples of such nanoscale wires are discussed in detail below, and include, for instance, semiconductor nanowires, carbon nanotubes, or the like. The nanoscale wires may also be straight, or kinked in some cases. In some embodiments, one or more of the nanoscale wires may form at least a portion of a transistor, such as a field-effect transistor, e.g., as is discussed in more detail below. The nanoscale wires may be distributed within the probe in any suitable configuration, for example, in an ordered array or randomly distributed. In some cases, the nanoscale wires are distributed such that an increasing concentration of nanoscale wires can be found towards the portion of the probe that is first inserted.

In some cases, some or all of the nanoscale wires are individually electronically addressable within the probe. For instance, in some cases, at least about 10%, at least about 20%, at least about 30%, at least about 40%, at least about 50%, at least about 60%, at least about 70%, at least about 80%, at least about 90%, or substantially all of the nanoscale wires may be individually electronically addressable. In some embodiments, an electrical property of a nanoscale wire can be individually determinable (e.g., being partially or fully resolvable without also including the electrical properties of other nanoscale wires), and/or such that the electrical property of a nanoscale wire may be individually controlled (for example, by applying a desired voltage or current to the nanoscale wire, for instance, without simultaneously applying the voltage or current to other nanoscale wires). In other embodiments, however, at least some of the nanoscale wires can be controlled within the same electronic circuit (e.g., by incorporating the nanoscale wires in series and/or in parallel), such that the nanoscale wires can still be electronically controlled and/or determined.

In various embodiments, more than one nanoscale wire may be present within the probe. The nanoscale wires may each independently be the same or different. For example, the probe may comprise at least 5 nanoscale wires, at least about 10 nanoscale wires, at least about 15 nanoscale wires, at least about 20 nanoscale wires, at least about 25 nanoscale wires, at least about 30 nanoscale wires, at least about 50 nanoscale wires, at least about 100 nanoscale wires, at least about 300 nanoscale wires, at least about 1000 nanoscale wires, etc.

In addition, in some embodiments, there may be a relatively high density of nanoscale wires within the probe, or at least a portion of the probe. The nanoscale wires may be distributed uniformly or non-uniformly on the probe. In some cases, the nanoscale wires may be distributed at an average density of at least about 5 wires/mm², at least about 10 wires/mm², at least about 30 wires/mm², at least about 50 wires/mm², at least about 75 wires/mm², at least about 100 wires/mm², at least about 300 wires/mm², at least about 500 wires/mm², at least about 750 wires/mm², at least about 1000 wires/mm², etc. In certain embodiments, the nanoscale wires are distributed such that the average separation between a nanoscale wire and its nearest neighboring nanoscale wire is less than about 2 mm, less than about 1 mm, less than about 500 micrometers, less than about 300 micrometers, less than about 100 micrometers, less than about 50 micrometers, less than about 30 micrometers, or less than about 10 micrometers.

Some or all of the nanoscale wires may be in electrical communication with one or more electrical connectors via one or more conductive pathways. The electrical connectors may be positioned on a portion of the probe that is not inserted into the tissue. The electrical connectors may be made out of any suitable material that allows transmission of an electrical signal. For example, the electrical connectors may comprise gold, silver, copper, aluminum, tantalum, titanium, nickel, tungsten, chromium, palladium, etc. In some cases, the electrical connectors have an average cross-section of less than about 10 micrometers, less than about 8 micrometers, less than about 6 micrometers, less than about 5 micrometers, less than about 4 micrometers, less than about 3 micrometers, less than about 2 micrometers, less than about 1 micrometer, etc.

In some embodiments, the electrical connectors can be used to determine a property of a nanoscale wire within the probe (for example, an electrical property or a chemical property as is discussed herein), and/or to direct an electrical signal to a nanoscale wire, e.g., to electrically stimulate cells proximate the nanoscale wire. The conductive pathways can form an electrical circuit that is internally contained within the probe, and/or that extends externally of the probe, e.g., such that the electrical circuit is in electrical communication with an external electrical system, such as a computer or a transmitter (for instance, a radio transmitter, a wireless transmitter, an Internet connection, etc.). Any suitable pathway conductive pathway may be used, for example, pathways comprising metals, semiconductors, conductive polymers, or the like.

Furthermore, more than one conductive pathway may be used in certain embodiments. For example, multiple conductive pathways can be used such that some or all of the nanoscale wires within the probe may be electronically individually addressable, as previously discussed. However, in other embodiments, more than one nanoscale wire may be addressable by a particular conductive pathway. In addition, in some cases, other electronic components may also be present within the probe, e.g., as part of a conductive pathway or otherwise forming part of an electrical circuit. Examples include, but are not limited to, transistors such as field-effect transistors or bipolar junction transistors, resistors, capacitors, inductors, diodes, integrated circuits, etc. In certain cases, some of these may also comprise nanoscale wires. For example, in some embodiments, two sets of electrical connectors and conductive pathways, and a nanoscale wire, may be used to define a transistor such as a field effect transistor, e.g., where the nanoscale wire defines the gate. As mentioned, the environment in and/or around the nanoscale wire can affect the ability of the nanoscale wire to function as a gate.

As mentioned, in various embodiments, one or more electrodes, electrical connectors, and/or conductive pathways may be positioned in electrical and/or physical communication with the nanoscale wires. These can be patterned to be in direct physical contact the nanoscale wire and/or there may be other materials that allow electrical communication to occur. Metals may be used due to their high conductance, e.g., such that changes within electrical properties obtained from the conductive pathway may be related to changes in properties of the nanoscale wire, rather than changes in properties of the conductive pathway. However, in other embodiments, other types of electrode materials are used, in addition or instead of metals.

A wide variety of metals may be used in various embodiments of the invention, for example in an electrode, electrical connector, conductive pathway, metal construct, polymer construct, etc. As non-limiting examples, the metals may include one or more of aluminum, gold, silver, copper, molybdenum, tantalum, titanium, nickel, tungsten, chromium, palladium, as well as any combinations of these and/or other metals. In some cases, the metal may be chosen to be one that is readily introduced, e.g., using techniques compatible with lithographic techniques. For example, in one set of embodiments, lithographic techniques such as e-beam lithography, photolithography, X-ray lithography, extreme ultraviolet lithography, ion projection lithography, etc. can be used to pattern or deposit one or more metals.

Additional processing steps can also be used to define or register the electrode, electrical connector, conductive pathway, metal construct, polymer construct, etc. in some cases. Thus, for example, the thickness of one of these may be less than about 5 micrometers, less than about 4 micrometers, less than about 3 micrometers, less than about 2 micrometers, less than about 1 micrometer, less than about 700 nm, less than about 600 nm, less than about 500 nm, less than about 300 nm, less than about 200 nm, less than about 100 nm, less than about 80 nm, less than about 50 nm, less than about 30 nm, less than about 10 nm, less than about 5 nm, less than about 2 nm, etc. The thickness of the electrode may also be at least about 10 nm, at least about 20 nm, at least about 40 nm, at least about 60 nm, at least about 80 nm, or at least about 100 nm. For example, the thickness of an electrode may be between about 40 nm and about 100 nm, between about 50 nm and about 80 nm.

In some embodiments, more than one metal may be used. The metals can be deposited in different regions or alloyed together, or in some cases, the metals may be layered on top of each other, e.g., layered on top of each other using various lithographic techniques. For example, a second metal may be deposited on a first metal, and in some cases, a third metal may be deposited on the second metal, etc. Additional layers of metal (e.g., fourth, fifth, sixth, etc.) can also be used in some embodiments. The metals may all be different, or in some cases, some of the metals (e.g., the first and third metals) may be the same. Each layer may independently be of any suitable thickness or dimension, e.g., of the dimensions described above, and the thicknesses of the various layers may independently be the same or different.

As mentioned, any nanoscale wire can be used in the probe. Non-limiting examples of suitable nanoscale wires include carbon nanotubes, nanorods, nanowires, organic and inorganic conductive and semiconducting polymers, metal nanoscale wires, semiconductor nanoscale wires (for example, formed from silicon), and the like. If carbon nanotubes are used, they may be single-walled and/or multi-walled, and may be metallic and/or semiconducting in nature. Other conductive or semiconducting elements that may not be nanoscale wires, but are of various small nanoscopic-scale dimension, also can be used in certain embodiments.

In general, a “nanoscale wire” (also known herein as a “nanoscopic-scale wire” or “nanoscopic wire”) generally is a wire or other nanoscale object, that at any point along its length, has at least one cross-sectional dimension and, in some embodiments, two orthogonal cross-sectional dimensions (e.g., a diameter) of less than 1 micrometer, less than about 500 nm, less than about 200 nm, less than about 150 nm, less than about 100 nm, less than about 70, less than about 50 nm, less than about 20 nm, less than about 10 nm, less than about 5 nm, than about 2 nm, or less than about 1 nm. In some embodiments, the nanoscale wire is generally cylindrical. In other embodiments, however, other shapes are possible; for example, the nanoscale wire can be faceted, i.e., the nanoscale wire may have a polygonal cross-section. The cross-section of a nanoscale wire can be of any arbitrary shape, including, but not limited to, circular, square, rectangular, annular, polygonal, or elliptical, and may be a regular or an irregular shape. The nanoscale wire can also be solid or hollow.

In some cases, the nanoscale wire has one dimension that is substantially longer than the other dimensions of the nanoscale wire. For example, the nanoscale wire may have a longest dimension that is at least about 1 micrometer, at least about 3 micrometers, at least about 5 micrometers, or at least about 10 micrometers or about 20 micrometers in length, and/or the nanoscale wire may have an aspect ratio (longest dimension to shortest orthogonal dimension) of greater than about 2:1, greater than about 3:1, greater than about 4:1, greater than about 5:1, greater than about 10:1, greater than about 25:1, greater than about 50:1, greater than about 75:1, greater than about 100:1, greater than about 150:1, greater than about 250:1, greater than about 500:1, greater than about 750:1, or greater than about 1000:1 or more in some cases.

In some embodiments, a nanoscale wire are substantially uniform, or have a variation in average diameter of the nanoscale wire of less than about 30%, less than about 25%, less than about 20%, less than about 15%, less than about 10%, or less than about 5%. For example, the nanoscale wires may be grown from substantially uniform nanoclusters or particles, e.g., colloid particles. See, e.g., U.S. Pat. No. 7,301,199, issued Nov. 27, 2007, entitled “Nanoscale Wires and Related Devices,” by Lieber, et al., incorporated herein by reference in its entirety. In some cases, the nanoscale wire may be one of a population of nanoscale wires having an average variation in diameter, of the population of nanowires, of less than about 30%, less than about 25%, less than about 20%, less than about 15%, less than about 10%, or less than about 5%.

In some embodiments, a nanoscale wire has a conductivity of or of similar magnitude to any semiconductor or any metal. The nanoscale wire can be formed of suitable materials, e.g., semiconductors, metals, etc., as well as any suitable combinations thereof. In some cases, the nanoscale wire will have the ability to pass electrical charge, for example, being electrically conductive. For example, the nanoscale wire may have a relatively low resistivity, e.g., less than about 10⁻³ Ohm m, less than about 10⁻⁴ Ohm m, less than about 10⁻⁶ Ohm m, or less than about 10⁻⁷ Ohm m. The nanoscale wire can, in some embodiments, have a conductance of at least about 1 microsiemens, at least about 3 microsiemens, at least about 10 microsiemens, at least about 30 microsiemens, or at least about 100 microsiemens.

The nanoscale wire can be solid or hollow, in various embodiments. As used herein, a “nanotube” is a nanoscale wire that is hollow, or that has a hollowed-out core, including those nanotubes known to those of ordinary skill in the art. As another example, a nanotube may be created by creating a core/shell nanowire, then etching away at least a portion of the core to leave behind a hollow shell. Accordingly, in one set of embodiments, the nanoscale wire is a non-carbon nanotube. In contrast, a “nanowire” is a nanoscale wire that is typically solid (i.e., not hollow). Thus, in one set of embodiments, the nanoscale wire may be a semiconductor nanowire, such as a silicon nanowire.

In one set of embodiment, a nanoscale wire may comprise or consist essentially of a metal. Non-limiting examples of potentially suitable metals include aluminum, gold, silver, copper, molybdenum, tantalum, titanium, nickel, tungsten, chromium, or palladium. In another set of embodiments, a nanoscale wire comprises or consists essentially of a semiconductor. Typically, a semiconductor is an element having semiconductive or semi-metallic properties (i.e., between metallic and non-metallic properties). An example of a semiconductor is silicon. Other non-limiting examples include elemental semiconductors, such as gallium, germanium, diamond (carbon), tin, selenium, tellurium, boron, or phosphorous. In other embodiments, more than one element may be present in the nanoscale wire as the semiconductor, for example, gallium arsenide, gallium nitride, indium phosphide, cadmium selenide, etc. Still other examples include a Group II-VI material (which includes at least one member from Group II of the Periodic Table and at least one member from Group VI, for example, ZnS, ZnSe, ZnSSe, ZnCdS, CdS, or CdSe), or a Group III-V material (which includes at least one member from Group III and at least one member from Group V, for example GaAs, GaP, GaAsP, InAs, InP, AlGaAs, or InAsP).

In certain embodiments, the semiconductor can be undoped or doped (e.g., p-type or n-type). For example, in one set of embodiments, a nanoscale wire may be a p-type semiconductor nanoscale wire or an n-type semiconductor nanoscale wire, and can be used as a component of a transistor such as a field effect transistor (“FET”). For instance, the nanoscale wire may act as the “gate” of a source-gate-drain arrangement of a FET, while metal leads or other conductive pathways (as discussed herein) are used as the source and drain electrodes.

In some embodiments, a dopant or a semiconductor may include mixtures of Group IV elements, for example, a mixture of silicon and carbon, or a mixture of silicon and germanium. In other embodiments, the dopant or the semiconductor may include a mixture of a Group III and a Group V element, for example, BN, BP, BAs, AN, AlP, AlAs, AlSb, GaN, GaP, GaAs, GaSb, InN, InP, InAs, or InSb. Mixtures of these may also be used, for example, a mixture of BN/BP/BAs, or BN/AlP. In other embodiments, the dopants may include alloys of Group III and Group V elements. For example, the alloys may include a mixture of AlGaN, GaPAs, InPAs, GaInN, AlGaInN, GaInAsP, or the like. In other embodiments, the dopants may also include a mixture of Group II and Group VI semiconductors. For example, the semiconductor may include ZnO, ZnS, ZnSe, ZnTe, CdS, CdSe, CdTe, HgS, HgSe, HgTe, BeS, BeSe, BeTe, MgS, MgSe, or the like. Alloys or mixtures of these dopants are also be possible, for example, (ZnCd)Se, or Zn(SSe), or the like. Additionally, alloys of different groups of semiconductors may also be possible, for example, a combination of a Group II-Group VI and a Group III-Group V semiconductor, for example, (GaAs)ₓ; (ZnS)₁₋ₓ. Other examples of dopants may include combinations of Group IV and Group VI elemnts, such as GeS, GeSe, GeTe, SnS, SnSe, SnTe, PbO, PbS, PbSe, or PbTe. Other semiconductor mixtures may include a combination of a Group I and a Group VII, such as CuF, CuCl, CuBr, Cut AgF, AgCl, AgBr, AgI, or the like. Other dopant compounds may include different mixtures of these elements, such as BeSiN₂, CaCN₂, ZnGeP₂, CdSnAs₂, ZnSnSb₂, CuGeP₃, CuSi₂P₃, Si₃N₄, Ge₃N₄, Al₂O₃, (Al, Ga, In)₂(S, Se, Te)₃, Al₂CO, (Cu, Ag)(Al, Ga, In, Tl, Fe)(S, Se, Te)₂ and the like.

The doping of the semiconductor to produce a p-type or n-type semiconductor may be achieved via bulk-doping in certain embodiments, although in other embodiments, other doping techniques (such as ion implantation) can be used. Many such doping techniques that can be used will be familiar to those of ordinary skill in the art, including both bulk doping and surface doping techniques. A bulk-doped article (e.g. an article, or a section or region of an article) is an article for which a dopant is incorporated substantially throughout the crystalline lattice of the article, as opposed to an article in which a dopant is only incorporated in particular regions of the crystal lattice at the atomic scale, for example, only on the surface or exterior. For example, some articles are typically doped after the base material is grown, and thus the dopant only extends a finite distance from the surface or exterior into the interior of the crystalline lattice. It should be understood that “bulk-doped” does not define or reflect a concentration or amount of doping in a semiconductor, nor does it necessarily indicate that the doping is uniform. “Heavily doped” and “lightly doped” are terms the meanings of which are clearly understood by those of ordinary skill in the art. In some embodiments, one or more regions comprise a single monolayer of atoms (“delta-doping”). In certain cases, the region may be less than a single monolayer thick (for example, if some of the atoms within the monolayer are absent). As a specific example, the regions may be arranged in a layered structure within the nanoscale wire, and one or more of the regions can be delta-doped or partially delta-doped.

Accordingly, in one set of embodiments, the nanoscale wires may include a heterojunction, e.g., of two regions with dissimilar materials or elements, and/or the same materials or elements but at different ratios or concentrations. The regions of the nanoscale wire may be distinct from each other with minimal cross-contamination, or the composition of the nanoscale wire can vary gradually from one region to the next. The regions may be both longitudinally arranged relative to each other, or radially arranged (e.g., as in a core/shell arrangement) on the nanoscale wire. Each region may be of any size or shape within the wire. The junctions may be, for example, a p/n junction, a p/p junction, an n/n junction, a p/i junction (where i refers to an intrinsic semiconductor), an n/i junction, an i/i junction, or the like. The junction can also be a Schottky junction in some embodiments. The junction may also be, for example, a semiconductor/semiconductor junction, a semiconductor/metal junction, a semiconductor/insulator junction, a metal/metal junction, a metal/insulator junction, an insulator/insulator junction, or the like. The junction may also be a junction of two materials, a doped semiconductor to a doped or an undoped semiconductor, or a junction between regions having different dopant concentrations. The junction can also be a defected region to a perfect single crystal, an amorphous region to a crystal, a crystal to another crystal, an amorphous region to another amorphous region, a defected region to another defected region, an amorphous region to a defected region, or the like. More than two regions may be present, and these regions may have unique compositions or may comprise the same compositions. As one example, a wire can have a first region having a first composition, a second region having a second composition, and a third region having a third composition or the same composition as the first composition. Non-limiting examples of nanoscale wires comprising heterojunctions (including core/shell heterojunctions, longitudinal heterojunctions, etc., as well as combinations thereof) are discussed in U.S. Pat. No. 7,301,199, issued Nov. 27, 2007, entitled “Nanoscale Wires and Related Devices,” by Lieber, et al., incorporated herein by reference in its entirety.

In some embodiments, the nanoscale wire is a bent or a kinked nanoscale wire. A kink is typically a relatively sharp transition or turning between a first substantially straight portion of a wire and a second substantially straight portion of a wire. For example, a nanoscale wire may have 1, 2, 3, 4, or 5 or more kinks. In some cases, the nanoscale wire is formed from a single crystal and/or comprises or consists essentially of a single crystallographic orientation, for example, a<110> crystallographic orientation, a <112> crystallographic orientation, or a<1120> crystallographic orientation. It should be noted that the kinked region need not have the same crystallographic orientation as the rest of the semiconductor nanoscale wire. In some embodiments, a kink in the semiconductor nanoscale wire may be at an angle of about 120° or a multiple thereof. The kinks can be intentionally positioned along the nanoscale wire in some cases. For example, a nanoscale wire may be grown from a catalyst particle by exposing the catalyst particle to various gaseous reactants to cause the formation of one or more kinks within the nanoscale wire. Non-limiting examples of kinked nanoscale wires, and suitable techniques for making such wires, are disclosed in International Patent Application No. PCT/US2010/050199, filed Sep. 24, 2010, entitled “Bent Nanowires and Related Probing of Species,” by Tian, et al., published as WO 2011/038228 on Mar. 31, 2011, incorporated herein by reference in its entirety.

In one set of embodiments, the nanoscale wire is formed from a single crystal, for example, a single crystal nanoscale wire comprising a semiconductor. A single crystal item may be formed via covalent bonding, ionic bonding, or the like, and/or combinations thereof. While such a single crystal item may include defects in the crystal in some cases, the single crystal item is distinguished from an item that includes one or more crystals, not ionically or covalently bonded, but merely in close proximity to one another.

In some embodiments, the nanoscale wires used herein are individual or free-standing nanoscale wires. For example, an “individual” or a “free-standing” nanoscale wire may, at some point in its life, not be attached to another article, for example, with another nanoscale wire, or the free-standing nanoscale wire may be in solution. This is in contrast to nanoscale features etched onto the surface of a substrate, e.g., a silicon wafer, in which the nanoscale features are never removed from the surface of the substrate as a free-standing article. This is also in contrast to conductive portions of articles which differ from surrounding material only by having been altered chemically or physically, in situ, i.e., where a portion of a uniform article is made different from its surroundings by selective doping, etching, etc. An “individual” or a “free-standing” nanoscale wire is one that can be (but need not be) removed from the location where it is made, as an individual article, and transported to a different location and combined with different components to make a functional device such as those described herein and those that would be contemplated by those of ordinary skill in the art upon reading this disclosure.

The nanoscale wire, in some embodiments, may be responsive to a property external of the nanoscale wire, e.g., a chemical property, an electrical property, a physical property, etc. Such determination may be qualitative and/or quantitative, and such determinations may also be recorded, e.g., for later use. For example, in one set of embodiments, the nanoscale wire may be responsive to voltage. For instance, the nanoscale wire may exhibits a voltage sensitivity of at least about 5 microsiemens/V; by determining the conductivity of a nanoscale wire, the voltage surrounding the nanoscale wire may thus be determined. In other embodiments, the voltage sensitivity can be at least about 10 microsiemens/V, at least about 30 microsiemens/V, at least about 50 microsiemens/V, or at least about 100 microsiemens/V. Other examples of electrical properties that can be determined include resistance, resistivity, conductance, conductivity, impendence, or the like.

As another example, a nanoscale wire may be responsive to a chemical property of the environment surrounding the nanoscale wire. For example, an electrical property of the nanoscale wire can be affected by a chemical environment surrounding the nanoscale wire, and the electrical property can be thereby determined to determine the chemical environment surrounding the nanoscale wire. As a specific non-limiting example, the nanoscale wires may be sensitive to pH or hydrogen ions. Further non-limiting examples of such nanoscale wires are discussed in U.S. Pat. No. 7,129,554, filed Oct. 31, 2006, entitled “Nanosensors,” by Lieber, et al., incorporated herein by reference in its entirety.

As a non-limiting example, the nanoscale wire may have the ability to bind to an analyte indicative of a chemical property of the environment surrounding the nanoscale wire (e.g., hydrogen ions for pH, or concentration for an analyte of interest), and/or the nanoscale wire may be partially or fully functionalized, i.e. comprising surface functional moieties, to which an analyte is able to bind, thereby causing a determinable property change to the nanoscale wire, e.g., a change to the resistivity or impedance of the nanoscale wire. The binding of the analyte can be specific or non-specific. Functional moieties may include simple groups, selected from the groups including, but not limited to, —OH, —CHO, —COOH, —SO₃H, —CN, —NH₂, —SH, —COSH, —COOR, halide; biomolecular entities including, but not limited to, amino acids, proteins, sugars, DNA, antibodies, antigens, and enzymes; grafted polymer chains with chain length less than the diameter of the nanowire core, selected from a group of polymers including, but not limited to, polyamide, polyester, polyimide, polyacrylic; a shell of material comprising, for example, metals, semiconductors, and insulators, which may be a metallic element, an oxide, an sulfide, a nitride, a selenide, a polymer and a polymer gel. A non-limiting example of a protein is PSA (prostate specific antigen), which can be determined, for example, by modifying the nanoscale wires by binding monoclonal antibodies for PSA (Ab1) thereto. See, e.g., U.S. Pat. No. 8,232,584, issued Jul. 31, 2012, entitled “Nanoscale Sensors,” by Lieber, et al., incorporated herein by reference in its entirety.

In some embodiments, a reaction entity may be bound to a surface of the nanoscale wire, and/or positioned in relation to the nanoscale wire such that the analyte can be determined by determining a change in a property of the nanoscale wire. The “determination” may be quantitative and/or qualitative, depending on the application, and in some cases, the determination may also be analyzed, recorded for later use, transmitted, or the like. The term “reaction entity” refers to any entity that can interact with an analyte in such a manner to cause a detectable change in a property (such as an electrical property) of a nanoscale wire. The reaction entity may enhance the interaction between the nanowire and the analyte, or generate a new chemical species that has a higher affinity to the nanowire, or to enrich the analyte around the nanowire. The reaction entity can comprise a binding partner to which the analyte binds. The reaction entity, when a binding partner, can comprise a specific binding partner of the analyte. For example, the reaction entity may be a nucleic acid, an antibody, a sugar, a carbohydrate or a protein. Alternatively, the reaction entity may be a polymer, catalyst, or a quantum dot. A reaction entity that is a catalyst can catalyze a reaction involving the analyte, resulting in a product that causes a detectable change in the nanowire, e.g. via binding to an auxiliary binding partner of the product electrically coupled to the nanowire. Another exemplary reaction entity is a reactant that reacts with the analyte, producing a product that can cause a detectable change in the nanowire. The reaction entity can comprise a shell on the nanowire, e.g. a shell of a polymer that recognizes molecules in, e.g., a gaseous sample, causing a change in conductivity of the polymer which, in turn, causes a detectable change in the nanowire.

The term “binding partner” refers to a molecule that can undergo binding with a particular analyte, or “binding partner” thereof, and includes specific, semi-specific, and non-specific binding partners as known to those of ordinary skill in the art. The term “specifically binds,” when referring to a binding partner (e.g., protein, nucleic acid, antibody, etc.), refers to a reaction that is determinative of the presence and/or identity of one or other member of the binding pair in a mixture of heterogeneous molecules (e.g., proteins and other biologics). Thus, for example, in the case of a receptor/ligand binding pair the ligand would specifically and/or preferentially select its receptor from a complex mixture of molecules, or vice versa. An enzyme would specifically bind to its substrate, a nucleic acid would specifically bind to its complement, an antibody would specifically bind to its antigen. Other examples include, nucleic acids that specifically bind (hybridize) to their complement, antibodies specifically bind to their antigen, and the like. The binding may be by one or more of a variety of mechanisms including, but not limited to ionic interactions, and/or covalent interactions, and/or hydrophobic interactions, and/or van der Waals interactions, etc.

The antibody may be any protein or glycoprotein comprising or consisting essentially of one or more polypeptides substantially encoded by immunoglobulin genes or fragments of immunoglobulin genes. Examples of recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

Antibodies exist as intact immunoglobulins or as a number of well characterized fragments produced by digestion with various peptidases. Thus, for example, pepsin digests an antibody below (i.e. toward the Fc domain) the disulfide linkages in the hinge region to produce F(ab)′₂, a dimer of Fab which itself is a light chain joined to VHCH1 by a disulfide bond. The F(ab)′₂ may be reduced under mild conditions to break the disulfide linkage in the hinge region thereby converting the (Fab)₂ dimer into an Fab′ monomer. The Fab′ monomer is essentially a Fab with part of the hinge region. While various antibody fragments are defined in terms of the digestion of an intact antibody, one of skill will appreciate that such fragments may be synthesized de novo either chemically, by utilizing recombinant DNA methodology, or by “phage display” methods. Non-limiting examples of antibodies include single chain antibodies, e.g., single chain Fv (scFv) antibodies in which a variable heavy and a variable light chain are joined together (directly or through a peptide linker) to form a continuous polypeptide.

Thus, in some embodiments, a property such as a chemical property and/or an electrical property can be determined, e.g., at a resolution of less than about 2 mm, less than about 1 mm, less than about 500 micrometers, less than about 300 micrometers, less than about 100 micrometers, less than about 50 micrometers, less than about 30 micrometers, or less than about 10 micrometers, etc., e.g., due to the average separation between a nanoscale wire and its nearest neighboring nanoscale wire. In addition, the property may be determined within the tissue in 3 dimensions in some instances, in contrast with many other techniques where only a surface of the biological tissue can be studied. Accordingly, very high resolution and/or 3-dimensional mappings of the property of the biological tissue can be obtained in some embodiments. Any suitable tissue may be studied, e.g., brain tissue, cardiac tissue, vascular tissue, muscle, cartilage, bone, liver tissue, pancreatic tissue, bladder tissue, airway tissues, bone marrow tissue, or the like.

In addition, in some cases, such properties can be determined and/or recorded as a function of time. Thus, for example, such properties can be determined at a time resolution of less than about 1 min, less than about 30 s, less than about 15 s, less than about 10 s, less than about 5 s, less than about 3 s, less than about 1 s, less than about 500 ms, less than about 300 ms, less than about 100 ms, less than about 50 ms, less than about 30 ms, less than about 10 ms, less than about 5 ms, less than about 3 ms, less than about 1 ms, etc.

In yet another set of embodiments, the biological tissue, and/or portions of the biological tissue, may be electrically stimulated using nanoscale wires present within the tissue. For example, all, or a subset of the electrically active nanoscale wires may be electrically stimulated, e.g., by using an external electrical system, such as a computer. Thus, for example, a single nanoscale wire, a group of nanoscale wires, or substantially all of the nanoscale wires can be electrically stimulated, depending on the particular application. In some cases, such nanoscale wires can be stimulated in a particular pattern, e.g., to cause cardiac or muscle cells to contract or beat in a particular pattern (for example, as part of a prosthetic or a pacemaker), to cause the firing of neurons with a particular pattern, to monitor the status of an implanted tissue within a subject, or the like.

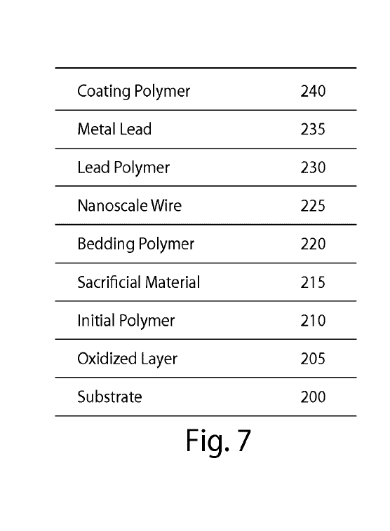

Another aspect of the present invention is generally directed to systems and methods for making and using such probes. Briefly, in one set of embodiments, a probe is constructed by assembling various polymers, metals, nanoscale wires, and other components together on a substrate. For example, lithographic techniques such as e-beam lithography, photolithography, X-ray lithography, extreme ultraviolet lithography, ion projection lithography, etc. may be used to pattern polymers, metals, etc. on the substrate, and nanoscale wires can be prepared separately then added to the substrate. After assembly, at least a portion of the substrate (e.g., a sacrificial material) may be removed, allowing the probe to be partially or completely removed from the substrate. The probe can, in some cases, be formed into a 3-dimensional structure, for example, spontaneously, or by folding or rolling the structure. Other materials may also be added to the probe, e.g., to help stabilize the structure, to add additional agents to enhance its biocompatibility, etc. The probe can be used in vivo, e.g., by implanting it in a subject, and/or in vitro, e.g., by seeding cells, etc. on the probe. In addition, in some cases, cells may initially be grown on the probe before the probe is implanted into a subject. A schematic diagram of the layers formed on the substrate in one embodiment is shown in FIG. 7. However, it should be understood that this diagram is illustrative only and is not drawn to scale, and not all of the layers shown in FIG. 7 are necessarily required in every embodiment of the invention.

The substrate (200 in FIG. 7) may be chosen to be one that can be used for lithographic techniques such as e-beam lithography or photolithography, or other lithographic techniques including those discussed herein. For example, the substrate may comprise or consist essentially of a semiconductor material such as silicon, although other substrate materials (e.g., a metal) can also be used. Typically, the substrate is one that is substantially planar, e.g., so that polymers, metals, and the like can be patterned on the substrate.

In some cases, a portion of the substrate can be oxidized, e.g., forming SiO₂ and/or Si₃N₄ on a portion of the substrate, which may facilitate subsequent addition of materials (metals, polymers, etc.) to the substrate. In some cases, the oxidized portion may form a layer of material on the substrate (205 in FIG. 7), e.g., having a thickness of less than about 5 micrometers, less than about 4 micrometers, less than about 3 micrometers, less than about 2 micrometers, less than about 1 micrometer, less than about 900 nm, less than about 800 nm, less than about 700 nm, less than about 600 nm, less than about 500 nm, less than about 400 nm, less than about 300 nm, less than about 200 nm, less than about 100 nm, etc.

In certain embodiments, one or more polymers can also be deposited or otherwise formed prior to depositing the sacrificial material. In some cases, the polymers may be deposited or otherwise formed as a layer of material (210 in FIG. 7) on the substrate. Deposition may be performed using any suitable technique, e.g., using lithographic techniques such as e-beam lithography, photolithography, X-ray lithography, extreme ultraviolet lithography, ion projection lithography, etc. In some cases, some or all of the polymers may be biocompatible and/or biodegradable. The polymers that are deposited may also comprise methyl methacrylate and/or poly(methyl methacrylate), in some embodiments. One, two, or more layers of polymer can be deposited (e.g., sequentially) in various embodiments, and each layer may independently have a thickness of less than about 5 micrometers, less than about 4 micrometers, less than about 3 micrometers, less than about 2 micrometers, less than about 1 micrometer, less than about 900 nm, less than about 800 nm, less than about 700 nm, less than about 600 nm, less than about 500 nm, less than about 400 nm, less than about 300 nm, less than about 200 nm, less than about 100 nm, etc.

Next, a sacrificial material may be deposited. The sacrificial material can be chosen to be one that can be removed without substantially altering other materials (e.g., polymers, other metals, nanoscale wires, etc.) deposited thereon. For example, in one embodiment, the sacrificial material may be a metal, e.g., one that is easily etchable. For instance, the sacrificial material can comprise germanium or nickel, which can be etched or otherwise removed, for example, using a peroxide (e.g., H₂O₂) or a nickel etchant (many of which are readily available commercially). In some cases, the sacrificial material may be deposited on oxidized portions or polymers previously deposited on the substrate. In some cases, the sacrificial material is deposited as a layer (e.g., 215 in FIG. 7). The layer can have a thickness of less than about 5 micrometers, less than about 4 micrometers, less than about 3 micrometers, less than about 2 micrometers, less than about 1 micrometer, less than about 900 nm, less than about 800 nm, less than about 700 nm, less than about 600 nm, less than about 500 nm, less than about 400 nm, less than about 300 nm, less than about 200 nm, less than about 100 nm, etc.

In some embodiments, a “bedding” polymer can be deposited, e.g., on the sacrificial material. The bedding polymer may include one or more polymers, which may be deposited as one or more layers (220 in FIG. 7). The bedding polymer can be used to support the nanoscale wires, and in some cases, partially or completely surround the nanoscale wires, depending on the application. For example, as discussed below, one or more nanoscale wires may be deposited on at least a portion of the uppermost layer of bedding polymer.

For instance, the bedding polymer can at least partially define a probe. In one set of embodiments, the bedding polymer may be deposited as a layer of material, such that portions of the bedding polymer may be subsequently removed. For example, the bedding polymer can be deposited using lithographic techniques such as e-beam lithography, photolithography, X-ray lithography, extreme ultraviolet lithography, ion projection lithography, etc., or using other techniques for removing polymer that are known to those of ordinary skill in the art. In some cases, more than one bedding polymer is used, e.g., deposited as more than one layer (e.g., sequentially), and each layer may independently have a thickness of less than about 5 micrometers, less than about 4 micrometers, less than about 3 micrometers, less than about 2 micrometers, less than about 1 micrometer, less than about 900 nm, less than about 800 nm, less than about 700 nm, less than about 600 nm, less than about 500 nm, less than about 400 nm, less than about 300 nm, less than about 200 nm, less than about 100 nm, etc. For example, in some embodiments, portions of the photoresist may be exposed to light (visible, UV, etc.), electrons, ions, X-rays, etc. (e.g., projected onto the photoresist), and the exposed portions can be etched away (e.g., using suitable etchants, plasma, etc.) to produce the pattern.

Accordingly, the bedding polymer may be formed into a particular pattern, e.g., in a grid, or in a pattern that suggests an endogenous probe, before or after deposition of nanoscale wires (as discussed in detail below), in certain embodiments of the invention. The pattern can be regular or irregular. For example, the bedding polymer can be formed into a pattern defining pore sizes such as those discussed herein. For instance, the polymer may have an average pore size of at least about 100 micrometers, at least about 200 micrometers, at least about 300 micrometers, at least about 400 micrometers, at least about 500 micrometers, at least about 600 micrometers, at least about 700 micrometers, at least about 800 micrometers, at least about 900 micrometers, or at least about 1 mm, and/or an average pore size of no more than about 1.5 mm, no more than about 1.4 mm, no more than about 1.3 mm, no more than about 1.2 mm, no more than about 1.1 mm, no more than about 1 mm, no more than about 900 micrometers, no more than about 800 micrometers, no more than about 700 micrometers, no more than about 600 micrometers, or no more than about 500 micrometers, etc.

Any suitable polymer may be used as the bedding polymer. In some cases, one or more of the polymers can be chosen to be biocompatible and/or biodegradable. In certain embodiments, one or more of the bedding polymers may comprise a photoresist. Photoresists can be useful due to their familiarity in use in lithographic techniques such as those discussed herein. Non-limiting examples of photoresists include SU-8, S1805, LOR 3A, poly(methyl methacrylate), poly(methyl glutarimide), phenol formaldehyde resin (diazonaphthoquinone/novolac), diazonaphthoquinone (DNQ), Hoechst AZ 4620, Hoechst AZ 4562, Shipley 1400-17, Shipley 1400-27, Shipley 1400-37, etc., as well as any others discussed herein.

In certain embodiments, one or more of the bedding polymers can be heated or baked, e.g., before or after depositing nanoscale wires thereon as discussed below, and/or before or after patterning the bedding polymer. For example, such heating or baking, in some cases, is important to prepare the polymer for lithographic patterning. In various embodiments, the bedding polymer may be heated to a temperature of at least about 30° C., at least about 65° C., at least about 95° C., at least about 150° C., or at least about 180° C., etc.

Next, one or more nanoscale wires (e.g., 225 in FIG. 7) may be deposited, e.g., on a bedding polymer on the substrate. Any of the nanoscale wires described herein may be used, e.g., n-type and/or p-type nanoscale wires, substantially uniform nanoscale wires (e.g., having a variation in average diameter of less than 20%), nanoscale wires having a diameter of less than about 1 micrometer, semiconductor nanowires, silicon nanowires, bent nanoscale wires, kinked nanoscale wires, core/shell nanowires, nanoscale wires with heterojunctions, etc. In some cases, the nanoscale wires are present in a liquid which is applied to the substrate, e.g., poured, painted, or otherwise deposited thereon. In some embodiments, the liquid is chosen to be relatively volatile, such that some or all of the liquid can be removed by allowing it to substantially evaporate, thereby depositing the nanoscale wires. In some cases, at least a portion of the liquid can be dried off, e.g., by applying heat to the liquid. Examples of suitable liquids include water or isopropanol.

In some cases, at least some of the nanoscale wires may be at least partially aligned, e.g., as part of the deposition process, and/or after the nanoscale wires have been deposited on the substrate. Thus, the alignment can occur before or after drying or other removal of the liquid, if a liquid is used. Any suitable technique may be used for alignment of the nanoscale wires. For example, the nanoscale wires can be aligned by passing or sliding substrates containing the nanoscale wires past each other (see, e.g., International Patent Application No. PCT/US2007/008540, filed Apr. 6, 2007, entitled “Nanoscale Wire Methods and Devices,” by Nam, et al., published as WO 2007/145701 on Dec. 21, 2007, incorporated herein by reference in its entirety), the nanoscale wires can be aligned using Langmuir-Blodgett techniques (see, e.g., U.S. patent application Ser. No. 10/995,075, filed Nov. 22, 2004, entitled “Nanoscale Arrays and Related Devices,” by Whang, et al., published as U.S. Patent Application Publication No. 2005/0253137 on Nov. 17, 2005, incorporated herein by reference in its entirety), the nanoscale wires can be aligned by incorporating the nanoscale wires in a liquid film or “bubble” which is deposited on the substrate (see, e.g., U.S. patent application Ser. No. 12/311,667, filed Apr. 8, 2009, entitled “Liquid Films Containing Nanostructured Materials,” by Lieber, et al., published as U.S. Patent Application Publication No. 2010/0143582 on Jun. 10, 2010, incorporated by reference herein in its entirety), or a gas or liquid can be passed across the nanoscale wires to align the nanoscale wires (see, e.g., U.S. Pat. No. 7,211,464, issued May 1, 2007, entitled “Doped Elongated Semiconductors, Growing Such Semiconductors, Devices Including Such Semiconductors, and Fabricating Such Devices,” by Lieber, et al.; and U.S. Pat. No. 7,301,199, issued Nov. 27, 2007, entitled “Nanoscale Wires and Related Devices,” by Lieber, et al., each incorporated herein by reference in its entirety). Combinations of these and/or other techniques can also be used in certain instances. In some cases, the gas may comprise an inert gas and/or a noble gas, such as nitrogen or argon.

In certain embodiments, a “lead” polymer is deposited (230 in FIG. 7), e.g., on the sacrificial material and/or on at least some of the nanoscale wires. The lead polymer may include one or more polymers, which may be deposited as one or more layers. The lead polymer can be used to cover or protect metal leads or other conductive pathways, which may be subsequently deposited on the lead polymer. In some embodiments, the lead polymer can be deposited, e.g., as a layer of material such that portions of the lead polymer can be subsequently removed, for instance, using lithographic techniques such as e-beam lithography, photolithography, X-ray lithography, extreme ultraviolet lithography, ion projection lithography, etc., or using other techniques for removing polymer that are known to those of ordinary skill in the art, similar to the bedding polymers previously discussed. However, the lead polymers need not be the same as the bedding polymers (although they can be), and they need not be deposited using the same techniques (although they can be). In some cases, more than one lead polymer may be used, e.g., deposited as more than one layer (for example, sequentially), and each layer may independently have a thickness of less than about 5 micrometers, less than about 4 micrometers, less than about 3 micrometers, less than about 2 micrometers, less than about 1 micrometer, less than about 900 nm, less than about 800 nm, less than about 700 nm, less than about 600 nm, less than about 500 nm, less than about 400 nm, less than about 300 nm, less than about 200 nm, less than about 100 nm, etc.

Any suitable polymer can be used as the lead polymer. In some cases, one or more of the polymers may be chosen to be biocompatible and/or biodegradable. For example, in one set of embodiments, one or more of the polymers may comprise poly(methyl methacrylate). In certain embodiments, one or more of the lead polymers comprises a photoresist, such as those described herein.

In certain embodiments, one or more of the lead polymers may be heated or baked, e.g., before or after depositing nanoscale wires thereon as discussed below, and/or before or after patterning the lead polymer. For example, such heating or baking, in some cases, is important to prepare the polymer for lithographic patterning. In various embodiments, the lead polymer may be heated to a temperature of at least about 30° C., at least about 65° C., at least about 95° C., at least about 150° C., or at least about 180° C., etc.

Next, a metal or other conductive material can be deposited (235 in FIG. 7), e.g., on one or more of the lead polymer, the sacrificial material, the nanoscale wires, etc. to form a metal lead or other conductive pathway. More than one metal can be used, which may be deposited as one or more layers. For example, a first metal may be deposited, e.g., on one or more of the lead polymers, and a second metal may be deposited on at least a portion of the first metal. Optionally, more metals can be used, e.g., a third metal may be deposited on at least a portion of the second metal, and the third metal may be the same or different from the first metal. In some cases, each metal may independently have a thickness of less than about 5 micrometers, less than about 4 micrometers, less than about 3 micrometers, less than about 2 micrometers, less than about 1 micrometer, less than about 900 nm, less than about 800 nm, less than about 700 nm, less than about 600 nm, less than about 500 nm, less than about 400 nm, less than about 300 nm, less than about 200 nm, less than about 100 nm, less than about 80 nm, less than about 60 nm, less than about 40 nm, less than about 30 nm, less than about 20 nm, less than about 10 nm, less than about 8 nm, less than about 6 nm, less than about 4 nm, or less than about 2 nm, etc., and the layers may be of the same or different thicknesses.

Any suitable technique can be used for depositing metals, and if more than one metal is used, the techniques for depositing each of the metals may independently be the same or different. For example, in one set of embodiments, deposition techniques such as sputtering can be used. Other examples include, but are not limited to, physical vapor deposition, vacuum deposition, chemical vapor deposition, cathodic arc deposition, evaporative deposition, e-beam PVD, pulsed laser deposition, ion-beam sputtering, reactive sputtering, ion-assisted deposition, high-target-utilization sputtering, high-power impulse magnetron sputtering, gas flow sputtering, or the like.

The metals can be chosen in some cases such that the deposition process yields a pre-stressed arrangement, e.g., due to atomic lattice mismatch, which causes the subsequent metal leads to warp or bend, for example, once released from the substrate. Although such processes were typically undesired in the prior art, in certain embodiments of the present invention, such pre-stressed arrangements may be used to cause the resulting probe to form a 3-dimensional structure, in some cases spontaneously, upon release from the substrate. However, it should be understood that in other embodiments, the metals may not necessary be deposited in a pre-stressed arrangement.

Examples of metals that can be deposited (stressed or unstressed) include, but are not limited to, aluminum, gold, silver, copper, molybdenum, tantalum, titanium, nickel, tungsten, chromium, palladium, as well as any combinations of these and/or other metals. For example, a chromium/palladium/chromium deposition process, in some embodiments, may form a pre-stressed arrangement that is able to spontaneously form a 3-dimensional structure after release from the substrate.

In certain embodiments, a “coating” polymer can be deposited (240 in FIG. 7), e.g., on at least some of the conductive pathways and/or at least some of the nanoscale wires. The coating polymer may include one or more polymers, which may be deposited as one or more layers. In some embodiments, the coating polymer may be deposited on one or more portions of a substrate, e.g., as a layer of material such that portions of the coating polymer can be subsequently removed, e.g., using lithographic techniques such as e-beam lithography, photolithography, X-ray lithography, extreme ultraviolet lithography, ion projection lithography, etc., or using other techniques for removing polymer that are known to those of ordinary skill in the art, similar to the other polymers previously discussed. The coating polymers can be the same or different from the lead polymers and/or the bedding polymers. In some cases, more than one coating polymer may be used, e.g., deposited as more than one layer (e.g., sequentially), and each layer may independently have a thickness of less than about 5 micrometers, less than about 4 micrometers, less than about 3 micrometers, less than about 2 micrometers, less than about 1 micrometer, less than about 900 nm, less than about 800 nm, less than about 700 nm, less than about 600 nm, less than about 500 nm, less than about 400 nm, less than about 300 nm, less than about 200 nm, less than about 100 nm, etc.

Any suitable polymer may be used as the coating polymer. In some cases, one or more of the polymers can be chosen to be biocompatible and/or biodegradable. For example, in one set of embodiments, one or more of the polymers may comprise poly(methyl methacrylate). In certain embodiments, one or more of the coating polymers may comprise a photoresist, e.g., as discussed herein.

In certain embodiments, one or more of the coating polymers can be heated or baked, e.g., before or after depositing nanoscale wires thereon as discussed below, and/or before or after patterning the coating polymer. For example, such heating or baking, in some cases, is important to prepare the polymer for lithographic patterning. In various embodiments, the coating polymer may be heated to a temperature of at least about 30° C., at least about 65° C., at least about 95° C., at least about 150° C., or at least about 180° C., etc.

After formation of the probe, some or all of the sacrificial material may then be removed in some cases. In one set of embodiments, for example, at least a portion of the sacrificial material is exposed to an etchant able to remove the sacrificial material. For example, if the sacrificial material is a metal such as nickel, a suitable etchant (for example, a metal etchant such as a nickel etchant, acetone, etc.) can be used to remove the sacrificial metal. Many such etchants may be readily obtained commercially. In addition, in some embodiments, the probe can also be dried, e.g., in air (e.g., passively), by using a heat source, by using a critical point dryer, etc.

In certain embodiments, upon removal of the sacrificial material, pre-stressed portions of the probe (e.g., metal leads containing dissimilar metals) can spontaneously cause the probe to adopt a 3-dimensional structure. In some cases, the probe may form a 3-dimensional structure as discussed herein. For example, the probe may have an open porosity of at least about 30%, at least about 40%, at least about 50%, at least about 60%, at least about 70%, at least about 75%, at least about 80%, at least about 85%, at least about 90%, at least about 95%, at least about 97, at least about 99%, at least about 99.5%, or at least about 99.8%. The probe may also have, in some cases, an average pore size of at least about 100 micrometers, at least about 200 micrometers, at least about 300 micrometers, at least about 400 micrometers, at least about 500 micrometers, at least about 600 micrometers, at least about 700 micrometers, at least about 800 micrometers, at least about 900 micrometers, or at least about 1 mm, and/or an average pore size of no more than about 1.5 mm, no more than about 1.4 mm, no more than about 1.3 mm, no more than about 1.2 mm, no more than about 1.1 mm, no more than about 1 mm, no more than about 900 micrometers, no more than about 800 micrometers, no more than about 700 micrometers, no more than about 600 micrometers, or no more than about 500 micrometers, etc.

However, in other embodiments, further manipulation may be needed to cause the probe to adopt a 3-dimensional structure, e.g., one with properties such as is discussed herein. For example, after removal of the sacrificial material, the probe may need to be rolled, curled, folded, creased, etc., or otherwise manipulated to form the 3-dimesional structure. Such manipulations can be done using any suitable technique, e.g., manually, or using a machine.

Other materials may be also added to the probe, e.g., before or after it forms a 3-dimensional structure, for example, to help stabilize the structure, to add additional agents to enhance its biocompatibility (e.g., growth hormones, extracellular matrix protein, Matrigeff, etc.), to cause it to form a suitable 3-dimension structure, to control pore sizes, etc. Non-limiting examples of such materials have been previously discussed above, and include other polymers, growth hormones, extracellular matrix protein, specific metabolites or nutrients, additional probe materials, or the like.

In addition, the probe can be interfaced in some embodiments with one or more electronics, e.g., an external electrical system such as a computer or a transmitter (for instance, a radio transmitter, a wireless transmitter, etc.). In some cases, electronic testing of the probe may be performed, e.g., before or after implantation into a subject. For instance, one or more of the metal leads may be connected to an external electrical circuit, e.g., to electronically interrogate or otherwise determine the electronic state or one or more of the nanoscale wires within the probe. Such determinations may be performed quantitatively and/or qualitatively, depending on the application, and can involve all, or only a subset, of the nanoscale wires contained within the probe, e.g., as discussed herein.

The following documents are incorporated herein by reference in their entireties: U.S. Pat. No. 7,211,464, issued May 1, 2007, entitled “Doped Elongated Semiconductors, Growing Such Semiconductors, Devices Including Such Semiconductors, and Fabricating Such Devices,” by Lieber, et al.; and U.S. Pat. No. 7,301,199, issued Nov. 27, 2007. Ser. No. 12/308,207, filed Ser. No. 10/588,833, filed Aug. 9, 2006, entitled “Nanostructures Containing Metal-Semiconductor Compounds,” by Lieber, et al., published as U.S. Patent Application Publication No. 2009/0004852 on Jan. 1, 2009; U.S. patent application Ser. No. 10/995,075, filed Nov. 22, 2004, entitled “Nanoscale Arrays, Robust Nanostructures, and Related Devices,” by Whang, et al., published as 2005/0253137 on Nov. 17, 2005; U.S. patent application Ser. No. 11/629,722, filed Dec. 15, 2006, entitled “Nanosensors,” by Wang, et al., published as U.S. Patent Application Publication No. 2007/0264623 on Nov. 15, 2007; International Patent Application No. PCT/US2007/008540, filed Apr. 6, 2007, entitled “Nanoscale Wire Methods and Devices,” by Lieber et al., published as WO 2007/145701 on Dec. 21, 2007; U.S. patent application Ser. No. ______ Dec. 9, 2008, entitled “Nanosensors and Related Technologies,” by Lieber, et al.; U.S. Pat. No. 8,232,584, issued Jul. 31, 2012, entitled “Nanoscale Sensors,” by Lieber, et al.; U.S. patent application Ser. No. 12/312,740, filed May 22, 2009, entitled “High-Sensitivity Nanoscale Wire Sensors,” by Lieber, et al., published as U.S. Patent Application Publication No. 2010/0152057 on Jun. 17, 2010; International Patent Application No. PCT/US2010/050199, filed Sep. 24, 2010, entitled “Bent Nanowires and Related Probing of Species,” by Tian, et al., published as WO 2011/038228 on Mar. 31, 2011; U.S. patent application Ser. No. 14/018,075, filed Sep. 4, 2013, entitled “Methods And Systems For Scaffolds Comprising Nanoelectronic Components,” by Lieber, et al.; and Int. Patent Application Serial No. PCT/US2013/055910, filed Aug. 19, 2013, entitled “Nanoscale Wire Probes,” by Lieber, et al. Also incorporated herein by reference in its entirety is U.S. Provisional Patent Application Ser. No. 61/911,294, filed Dec. 3, 2013, entitled “Nanoscale Wire Probes for the Brain and other Applications,” by Lieber, et al.

The following examples are intended to illustrate certain embodiments of the present invention, but do not exemplify the full scope of the invention.

### Example 1

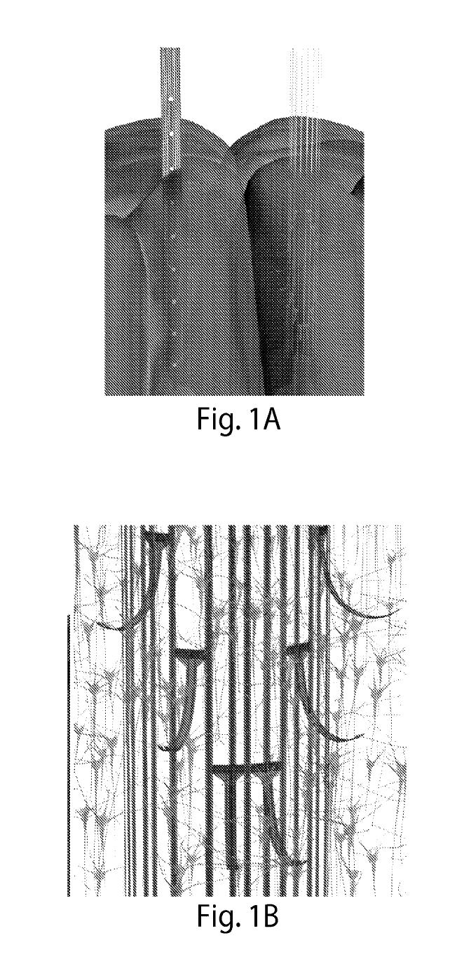

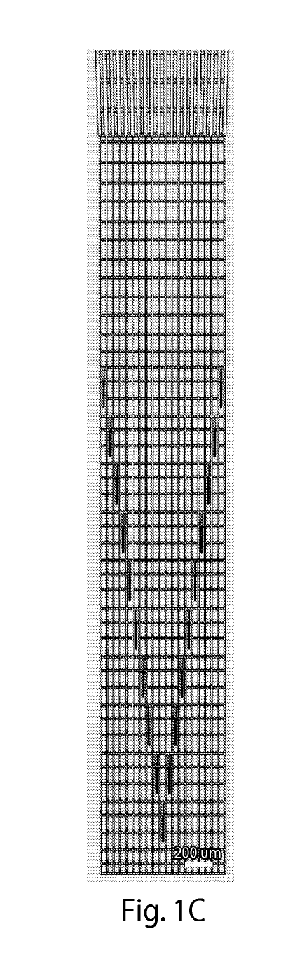

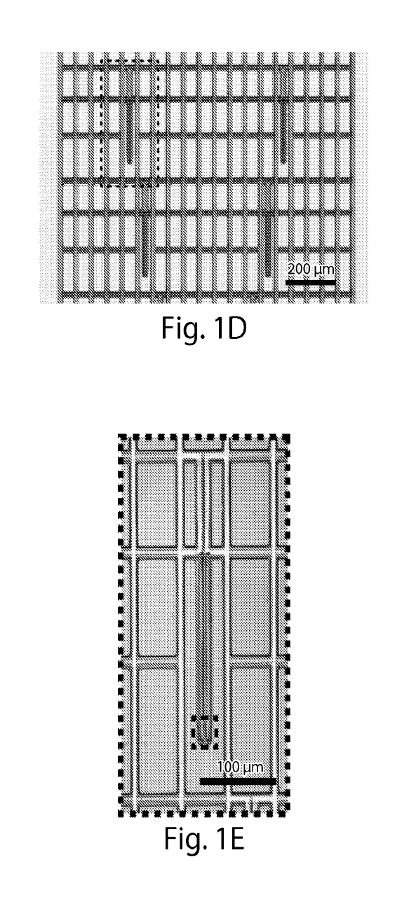

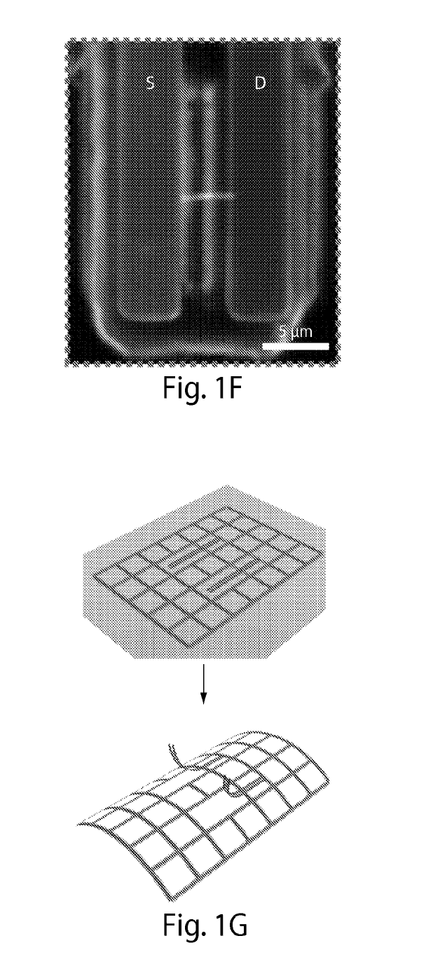

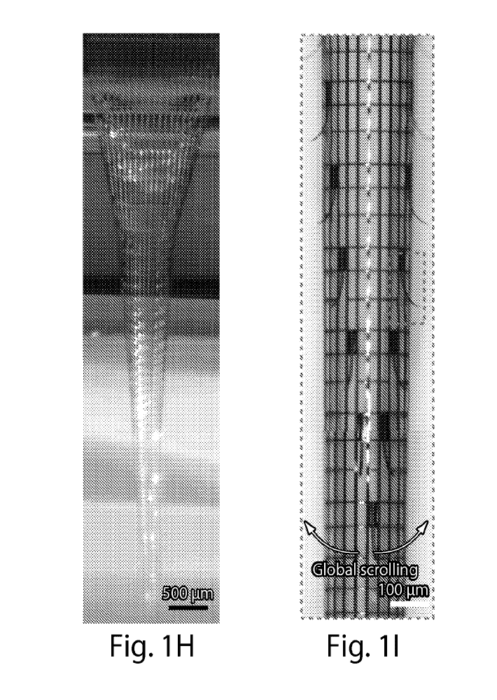

This example describes the preparation of a probe comprising nanoscale wires, for use as a brain implant. FIG. 1A is a schematic of the probe implanted into the brain, as compared with a conventional silicon probe. The nanowire probe demonstrates lower effective bending stiffness and an aporous structure that allows the probe and the brain tissue (e.g., neurons) to interpenetrate into each other, sub-cellular feature sizes, and a probe geometry that can facilitate the implantation procedure and optimize the probe-tissue interface. The nanowires are also distributed in three dimensions within the probe. FIG. 1B is a closer view of the probe, showing interpenetrating neurons. The open structure of the nanoscale wire allows for neurons and other brain cells to interpenetrate into the probe.

The probes are fabricated on a carrier wafer of a sacrificial layer of nickel, as is shown in FIG. 1C. These were fabricated using techniques such as photolithography which are known to those of ordinary skill in the art. FIG. 1D is a zoom-in of FIG. 1C, showing represent positive and negative bending areas, while FIG. 1E is a zoom-in of an individual nanoscale wire within the probe. FIG. 1F is a dark field image of the nanowire area of the probe. The nanowires are located at the end of the bend-up arms. The source and drain electrodes are well passivated by two layers of SU8. The channel region of the nanowires is suspended, which can increase the sensitivity of the devices.

FIG. 1G is a schematic diagram illustrating bending of the probe after fabrication. Built-in strain near the probe along the short axis of the probe can be used to shape the probe into a tubular structure once released from the sacrificial layer. This can be used to ensure that the probe is formed in a controlled manner and most of the nanowires are exposed at the surface of the probe. FIG. 1H shows a photograph of a probe released by etching away the sacrificial layer, suspended in buffer. FIG. 1I shows a zoom-in of the device area of the probe. Both global bending and local bending of the probe are illustrated.

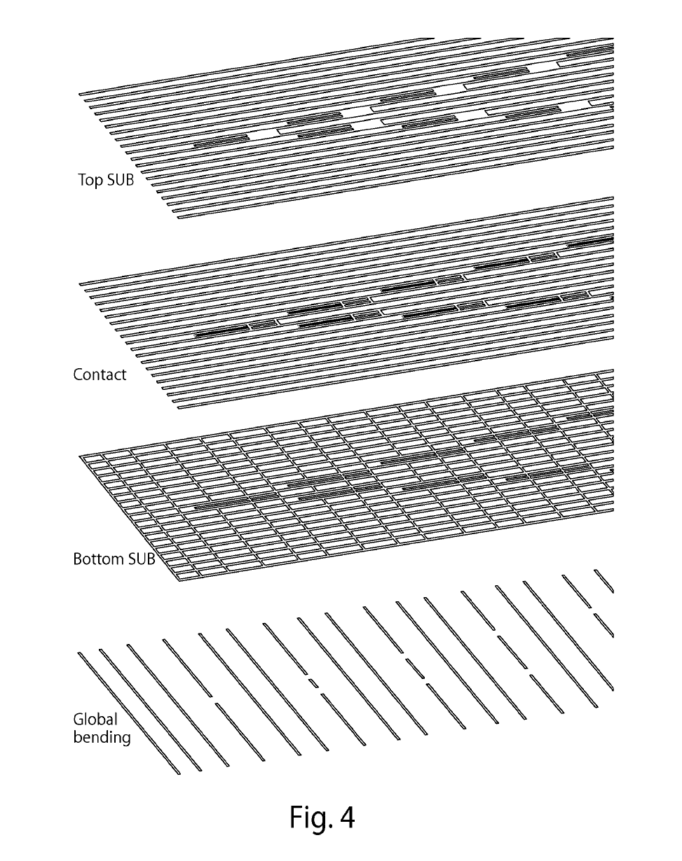

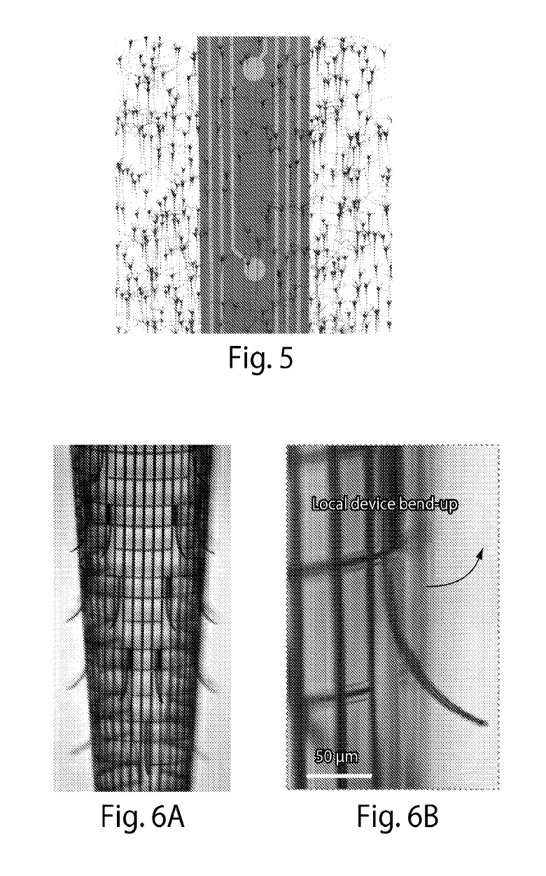

FIG. 4 is a schematic diagram showing the main layers used to form the probe. The contact layer was made of chromium/palladium/gold layers (1.5 nm/100 nm/200 nm) and the global bending layer was made of chromium/palladium layers (20 nm/80 nm). These were layered together on a sacrificial layer of nickel (100 nm thick) using photolithography or similar techniques to from the nanowire. After etching from the sacrificial layer using a nickel etchant, the layers were allowed to roll into a generally cylindrical probe driven by built-in strain from these materials. FIG. 6A shows a different probe design with more nanowires and a higher density of nanowires, while FIG. 6B shows an individual bent-up nanowire on a probe.

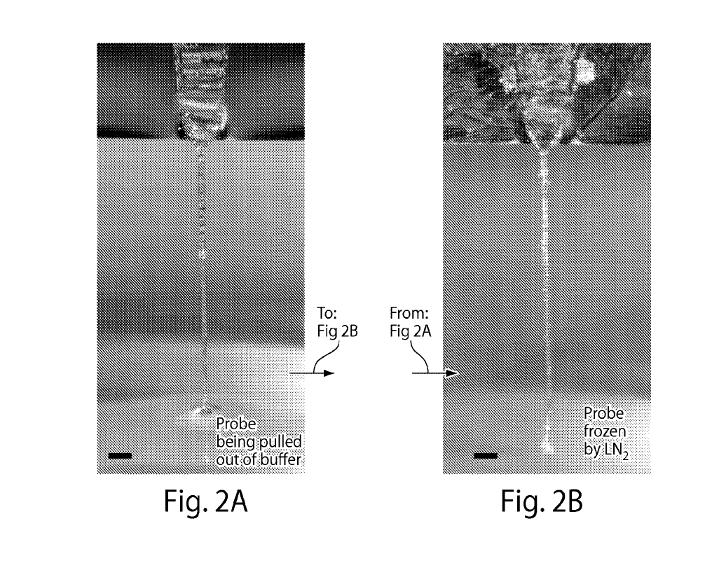

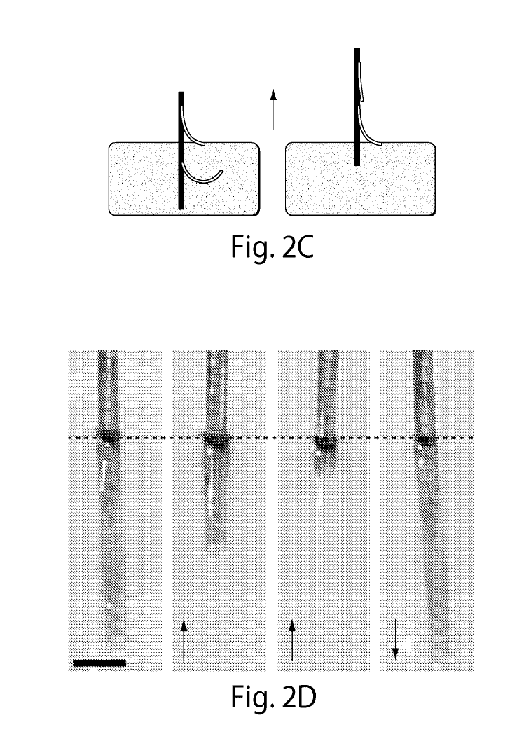

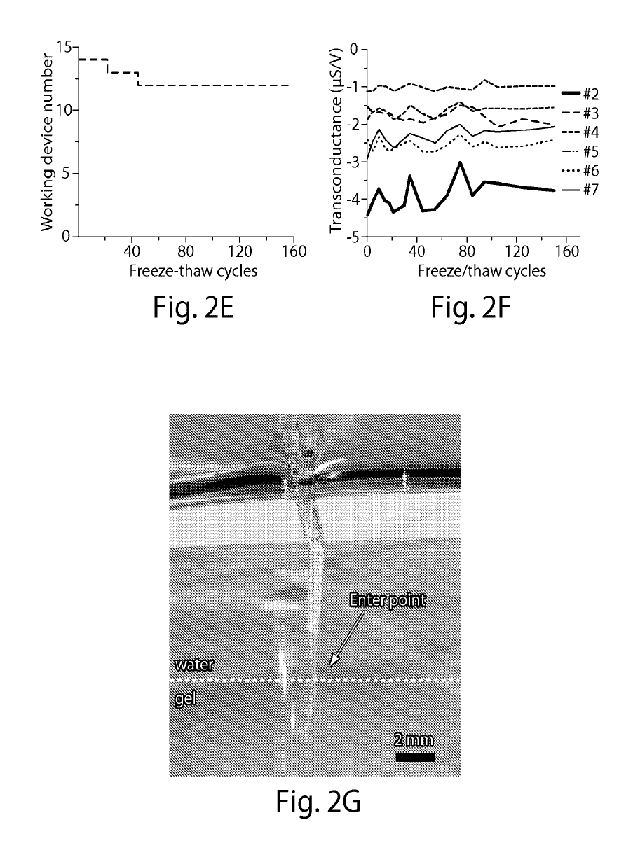

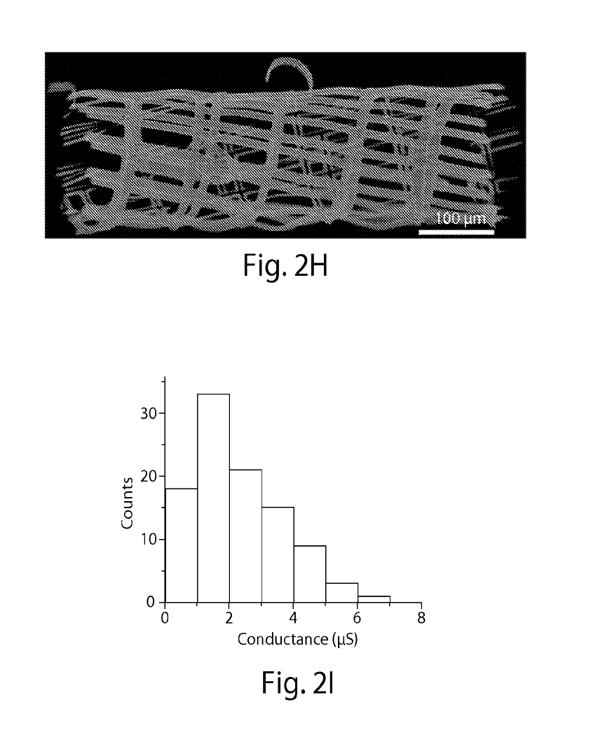

After formation, the probe was inserted into saline buffer (phosphate-buffered saline, physiological concentration) and slowly pulled out, as is shown in FIG. 2A. The built-in strain guided the probe to form a tubular structure. The working region of the probe (near the tip) had a diameter of about 200 micrometers, while the overall probe had a length of about 10 nm and a diameter of about 200 micrometers. The probe was then dipped in liquid nitrogen (LN2) (FIG. 2B), and became rigid enough to sustain penetration of the probe, e.g., through the brain tissue, agarose gel, tissue, etc. Repeated dipping in buffer and LN2 can be used in some cases to form extra coatings on the probe, which may be used to further enhance the strength of the probe.

FIG. 2C shows a schematic of bent-up portions of the device folding due to surface tension. While the probe was pulled out of the buffer, the bent-up portions were forced to the surface of the probe by the surface tension of the buffer. Once the restraining force disappears, the built-in strain will redeploy and bend up the device again, as is shown in FIG. 2D.

The probes may be fairly durable. For example, in one experiment, a probe with 14 nanowires was frozen and thawed for over 150 times. The survival number versus cycle number is plotted in FIG. 2E, showing that most of the nanowires were still functional after repeated freeze-thaw cycles. Similarly, FIG. 2F shows the transconductance of 7 devices out of the 14 devices in E versus cycle number, showing that the nanowires were not significantly changed after repeated freeze-thaw cycles.

FIG. 2G shows a photograph showing a probe partially inserted into the agarose gel (which can be used as a model of biological tissue). FIG. 2H is a 3D reconstruction of part of the probe embedded in the agarose gel using confocal microscopy. FIG. 2I is a histogram of the conductance of 100 nanowires after being subjected to a freeze-insert process, showing that most of the nanowires were not significantly affected by this process.

### Example 2

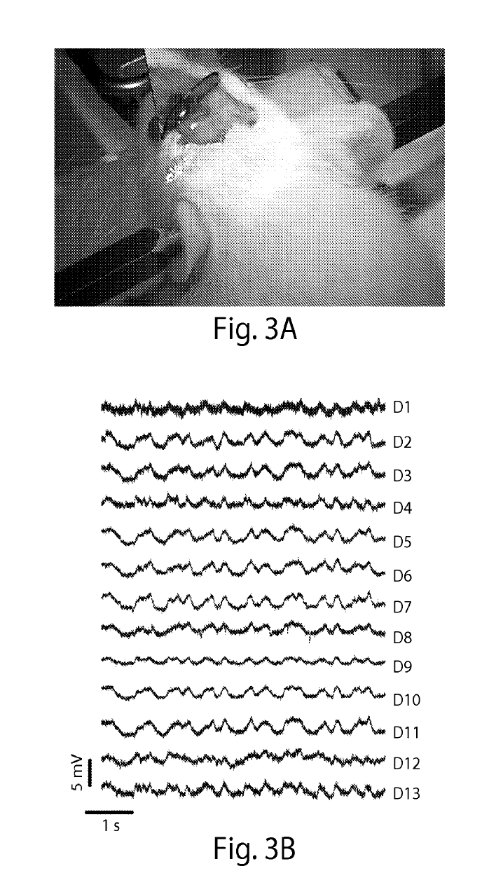

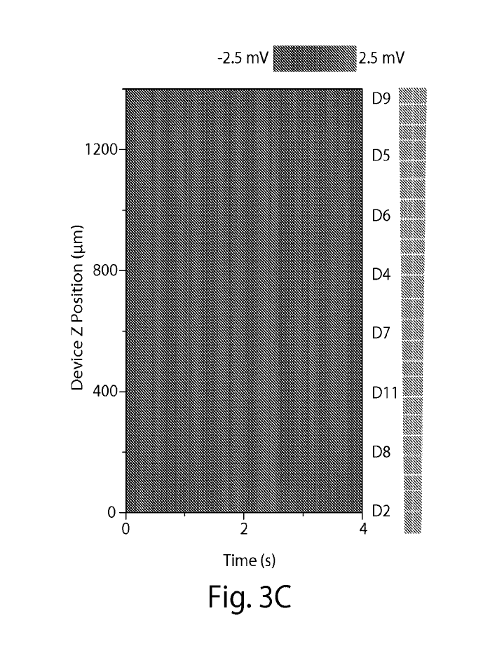

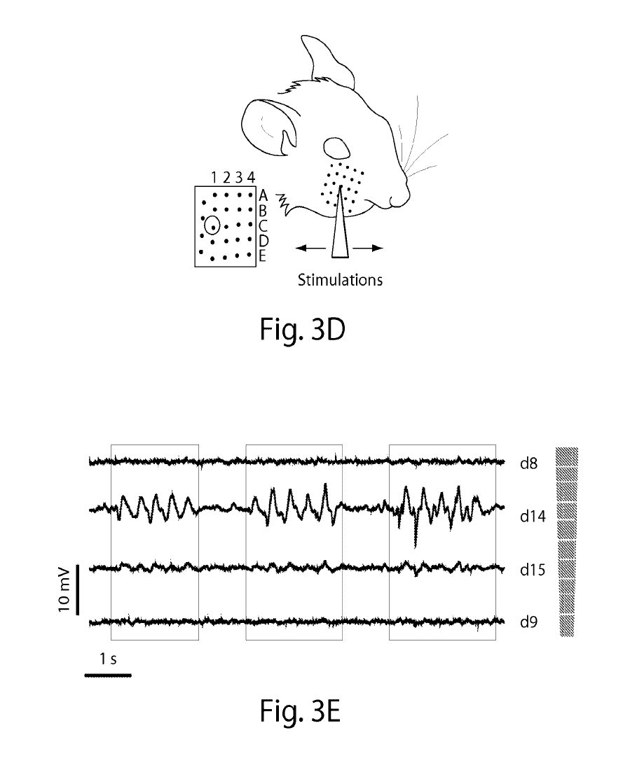

This example illustrates the recording of local field potentials (LFP) from the rat brain using a probe as discussed herein. FIG. 3A is a photograph of a typical rat brain surgery; the probe is implanted in its brain. The probe was dipped in saline followed by liquid nitrogen, as previously discussed, prior to insertion into the brain. After insertion, the probe warmed up to the body temperature of the rat.

FIG. 3B shows 13 channels of LFP recording. The recording by the probe was observed to be highly multiplexed. FIG. 3C shows a contour plot of the brain activity along Z direction mapped by 8 out of the 13 devices in FIG. 3B. The relative position of the 8 devices on the probe is also schematically illustrated on the right. FIGS. 3D and 3E show a demonstration of recordings from the probe correlated with rat behavior. The probe was inserted into the barrel cortex area in the somatosensory cortex, which is linked with the whiskers of the rat. It is shown that the recorded LFP correlates well with the stimulation to the whiskers; for example, stimulation of one whisker may correspond with an increase in activity in one location (as determined by one nanowire), without increasing activity in other areas; thus, for example, stimulation of a single whisker may trigger activity in only one of the nanowires. Recordings from 4 neighboring devices are shown in FIG. 3E.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method of inserting a probe, comprising:
coating at least a portion of a probe with a liquid, the probe comprising a polymer and an electrical network comprising at least one nanoscale wire;
exposing the probe to a temperature below the freezing point of the liquid and below the glass transition temperature of the polymer, whereby the liquid freezes; and
inserting the probe into biological tissue.

2. The method of claim 1, wherein the liquid is an aqueous liquid.

3. The method of any one of claim 1 or 2, wherein the liquid is saline.

4. The method of any one of claims 1-3, wherein exposing the probe to a temperature below the freezing point of the liquid and below the glass transition temperature of the polymer comprises exposing the probe to liquid nitrogen.

5. The method of any one of claims 1-4, wherein exposing the probe to a temperature below the freezing point of the liquid and below the glass transition temperature of the polymer comprises exposing the probe to a temperature of about −196° C. or less.

6. The method of any one of claims 1-5, wherein the biological tissue is a brain.

7. The method of any one of claims 1-6, wherein the biological tissue is human.

8. The method of any one of claims 1-7, wherein the biological tissue is alive.

9. The method of any one of claims 1-8, wherein coating at least a portion of the probe with the liquid comprises inserting the probe into a pool of the liquid.

10. The method of any one of claims 1-9, wherein coating at least a portion of the probe with the liquid comprises submerging at least a portion of the probe into the liquid.

11. The method of any one of claims 1-10, wherein coating at least a portion of the probe with the liquid causes a cross-sectional area defined by the probe, relative to a direction of insertion, to decrease by at least about 25%.

12. The method of any one of claims 1-11, further comprising determining an electrical property of at least one nanoscale wire of the electrical network after insertion of the probe.

13. The method of any one of claims 1-12, further comprising connecting the probe to an electrical apparatus after insertion of the probe.

14. The method of any one of claims 1-13, wherein the probe is connected to an electrical apparatus prior to insertion of the probe.

15. The method of any one of claims 1-14, wherein at least some of the nanoscale wires has a diameter of less than about 1 micrometer.

16. The method of any one of claims 1-15, wherein at least some of the nanoscale wires have a variation in average diameter of less than about 20%.

17. The method of any one of claims 1-16, wherein at least some of the nanoscale wires form part of a field effect transistor.

18. The method of any one of claims 1-17, wherein at least some of the nanoscale wires are responsive to an electrical property external to the nanoscale wire.

19. The method of any one of claims 1-18, wherein after insertion of the probe into biological tissue, the liquid thaws.

20. The method of any one of claims 1-19, wherein after insertion of the probe into biological tissue, the probe warms in temperature to a temperature greater than the glass transition temperature of the polymer.

21. The method of any one of claims 1-20, comprising configuring the probe from a first configuration into a second configuration prior to exposing the probe to a temperature below the freezing point of the liquid and below the glass transition temperature of the polymer, wherein after insertion of the probe into the biological tissue, the probe at least partially returns to the first configuration.

22. A method of inserting a probe, comprising:
providing an electrically-sensing probe comprising a polymer;
decreasing a cross-sectional area defined by the probe, relative to a direction of insertion, by at least about 25%;
exposing the polymer to a temperature below the glass transition temperature of the polymer; and
inserting the probe into a biological tissue.

23. The method of claim 22, wherein decreasing the cross-sectional area comprises exposing at least a portion of the probe to a liquid having a surface tension that causes the cross-sectional area to decrease by at least about 25% upon removal of the probe from the liquid.

24. The method of claim 23, wherein the liquid is an aqueous liquid.

25. The method of any one of claim 23 or 24, wherein the liquid is saline.

26. The method of any one of claims 23-25, wherein exposing at least a portion of the probe to the liquid comprises inserting the probe into a pool of the liquid.

27. The method of any one of claims 23-26, wherein exposing at least a portion of the probe to the liquid comprises submerging at least a portion of the probe into the liquid.

28. The method of any one of claims 22-27, wherein exposing the polymer to a temperature below the glass transition temperature of the polymer comprises exposing the probe to liquid nitrogen.

29. The method of any one of claims 22-28, wherein exposing the polymer to a temperature below the glass transition temperature of the polymer comprises exposing the probe to a temperature of about −196° C. or less.

30. The method of any one of claims 22-29, comprising decreasing the cross-sectional area by at least about 50%.

31. The method of any one of claims 22-30, comprising decreasing the cross-sectional area by at least about 75%.

32. The method of any one of claims 22-31, wherein the probe expands in cross-sectional area by at least about 10% after insertion into the biological tissue and exposure of the polymer to a temperature above the glass transition temperature of the polymer.

33. The method of any one of claims 22-32, wherein the biological tissue is a brain.

34. The method of any one of claims 22-33, wherein the biological tissue is human.

35. The method of any one of claims 22-34, further comprising determining an electrical property of the biological tissue after insertion of the probe.

36. The method of any one of claims 22-35, wherein the probe comprises a nanoscale wire.

37. The method of claim 36, wherein the nanoscale wire has a diameter of less than about 1 micrometer.

38. The method of any one of claim 36 or 37, wherein the nanoscale wire has a variation in average diameter of less than about 20%.

39. The method of claim 36-38, wherein the nanoscale wire form part of a field effect transistor.

40. The method of claim 36-39, wherein the nanoscale wire is responsive to an electrical property external to the nanoscale wire.

41. The method of any one of claims 22-40, wherein after insertion of the probe into biological tissue, the probe warms in temperature to a temperature greater than the glass transition temperature of the polymer.

42. The method of any one of claims 22-41, comprising configuring the probe from a first configuration into a second configuration prior to exposing the polymer to a temperature below the glass transition temperature of the polymer, wherein after insertion of the probe into the biological tissue, the probe at least partially returns to the first configuration.

43. A composition, comprising:
an electrical network comprising nanoscale wires, wherein at least a portion of the electrical network is coated with a solid or a liquid having a melting point below 25° C., and wherein the electrical network is at a temperature of about −196° C. or less.

44. The composition of claim 43, wherein the solid or liquid is aqueous.

45. The composition of any one of claim 43 or 44, wherein the solid or liquid is saline.

46. The composition of any one of claims 43-45, wherein at least some of the nanoscale wires comprise a semiconductor.

47. The composition of any one of claims 43-46, wherein at least some of the nanoscale wires comprise silicon.

48. The composition of any one of claims 43-47, wherein at least some of the nanoscale wires has a diameter of less than about 1 micrometer.

49. The composition of any one of claims 43-48, wherein at least some of the nanoscale wires have a variation in average diameter of less than about 20%.

50. The composition of any one of claims 43-49, wherein at least some of the nanoscale wires form part of a field effect transistor.

51. The composition of any one of claims 43-50, wherein at least some of the nanoscale wires are responsive to an electrical property external to the nanoscale wire.

52. The composition of any one of claims 43-51, wherein the coating is solid.

53. The composition of any one of claims 43-51, wherein the coating is liquid.

54. A method of inserting a probe, comprising:
coating at least a portion of a probe with a biocompatible fluid;
freezing at least a portion of the fluid coating on the probe; and
inserting the probe into biological tissue.
